(19) United States
(12) Patent Application Publication
(10) Pub. No.: US 2020/0237931 A1
(43) Pub. Date: Jul. 30, 2020

(54) MOLECULES TARGETING SURVIVAL MOTOR NEURON 2

(71) Applicant: Oxford University Innovation Limited, Oxford (GB)

(72) Inventors: Audrey Marie WINKELSAS, Bethesda, MD (US); Kenneth Henry FISCHBECK, Bethesda, MD (US); Christopher GRUNSEICH, Derwood, MD (US); George Geltmyier HARMISON, II, Silver Springs, MD (US)

(21) Appl. No.: 16/750,531

(22) Filed: Jan. 23, 2020

Related U.S. Application Data
(60) Provisional application No. 62/796,748, filed on Jan. 25, 2019.

(51) Int. Cl. A61K 48/00; C12N 15/113; A61P 21/00

(52) U.S. Cl. CPC A61K 48/0066; A61K 9/0019; A61P 21/00; C12N 15/113

(57) ABSTRACT

The present disclosure relates to agents, such as antisense oligonucleotides (oligomers) capable of binding to 5′ untranslated region (5′UTR) sequence of survival motor neuron 2 (SMN2) transcripts, and thereby cause the cell to increase the production of SMN2 mRNA and total SMN protein.

### CROSS-REFERENCE TO RELATED APPLICATIONS

This application claims priority to U.S. Provisional Patent Application Ser. No. 62/796,748 entitled “MOLECULES TARGETING SMN2” filed on Jan. 25, 2019, the entirety of which is hereby incorporated by reference.

### SEQUENCE LISTING

This application contains a sequence listing filed in electronic form as an ASCII.txt file entitled “8251010_ST25.txt” created on Jan. 17, 2020. The content of the sequence listing is incorporated herein in its entirety.

### FIELD OF DISCLOSURE

The present disclosure relates to agents, such as antisense oligonucleotides capable of binding to the 5′ untranslated region (5′UTR) sequence of survivor motor neuron 2 (SMN2) transcript, and thereby causing the cell to increase the production of SMN 1 and 2 mRNA and total SMN protein in cells. The present disclosure further relates to methods for the treatment of pathological conditions resulting from a deficiency in SMN2 in a cell or tissues of a human or animal subject.

### BACKGROUND

Spinal muscular atrophy (SMA) is a neuromuscular disorder caused by deletion of or mutations in the survival motor neuron 1 (SMN1) gene (Lefebvre et al., Cell. 80:155-165, 1995). SMN1 encodes the survival motor neuron protein (SMN), which is known to be involved in small nuclear ribonucleoprotein (snRNP) assembly, axonal mRNA transport, and neuromuscular junction maturation (Fischer et al., Cell. 90:1023-1029, 1997; Rage et al., RNA. 19:1755-1766, 2013; Kariya et al., J Clin Invest. 124:785-800, 2014). Low levels of SMN protein lead to the degeneration of motor neurons and subsequent muscle weakness in SMA. A second gene, SMN2, is highly homologous to SMN1, but a C-to-T transition in exon 7 results in exon skipping in a majority of transcripts (Lorson et al., Proc Natl Acad Sci USA. 96:6307-6311, 1999). As a result, only about 10 to 20 percent of SMN2 transcripts encode the fully functional SMN protein. The other 80 to 90 percent of protein produced from SMN2 (a truncated protein termed SMNΔ7) is unstable and quickly degraded.

The current FDA approved therapeutic for SMA, nusinersen, is an antisense oligonucleotide that promotes exon 7 inclusion (“splice-switching”) in the SMN2 transcript. This approach is very successful but has a ceiling effect that is dictated by the abundance of SMN2 transcripts in a cell. Increasing the total pool of SMN2 transcripts, or increasing the translational efficiency of these transcripts, are strategies to overcome the ceiling effect associated with the splice-switching approach.

Outside the region surrounding the exon 7 splicing event, the SMN2 transcript has been relatively unstudied. The 5′ and 3′ untranslated regions (UTRs) of genes contain cis-regulatory elements that modulate transcript stability and translational efficiency. The contribution of regulatory features in the 5′UTR to SMN2 expression, and the targetability of these features, is currently unknown.

### SUMMARY

One aspect of the disclosure encompasses embodiments of an oligonucleotide having between about 17 to about 35 nucleotides in length, wherein said oligonucleotide can comprise a base sequence complementary to at least 10 consecutive bases of the nucleotide sequence SEQ ID NO: 1, or a pharmaceutically acceptable salt thereof.

In some embodiments of this aspect of the disclosure, the oligonucleotide can be an antisense oligonucleotide.

In some embodiments of this aspect of the disclosure, the oligonucleotide can be 20 to 30 nucleotides in length.

In some embodiments of this aspect of the disclosure, the base sequence can be complementary to at least 12-20 consecutive bases of the nucleotide sequence SEQ ID NO: 1.

In some embodiments of this aspect of the disclosure, the base sequence can be complementary to all of the nucleotide sequence SEQ ID NO: 1.

In some embodiments of this aspect of the disclosure, the at least one sugar moiety, at least one phosphate bond moiety, or at least one sugar moiety and at least one phosphate bond moiety of the oligonucleotide can be modified.

In some embodiments of this aspect of the disclosure, (i) the modified sugar moiety can be a ribose wherein the —OH group at the 2′-position is substituted with a group selected from the group consisting of OR, R, R′OR, SH, SR, NH₂, NHR, NR₂, N₃, CN, F, Cl, Br and I (wherein R is an alkyl or an aryl group, and R′ represents an alkylene group or 2′-F-ANA or (ii) the modified sugar moiety can be a constrained ethyl (cET), an ethylene bridge nucleic acid (ENA), or a locked-nucleic acid (LNA) modification.

In some embodiments of this aspect of the disclosure, the modified sugar moiety can be a ribose wherein —OH group at the 2′-position is substituted with 2′-O-(2-methoxyethyl) (2-MOE) or 2′-O-methyl (2′-OMe).

In some embodiments of this aspect of the disclosure, the modified phosphate bond moiety can be selected from the group consisting of a phosphorothioate bond, a phosphorodithioate bond, an alkylphosphonate bond, a phosphoroamidate bond, and a boranophosphate bond.

In some embodiments of this aspect of the disclosure, the antisense oligomer can comprise at least one morpholino ring.

In some embodiments of this aspect of the disclosure, the oligonucleotide can be a morpholino oligomer or a phosphorodiamidate morpholino oligomer.

In some embodiments of this aspect of the disclosure, the intemucleoside linkages of the contiguous nucleotide sequence can be phosphorothioate internucleoside linkages.

In some embodiments of this aspect of the disclosure, the oligonucleotide can comprise a sequence selected from the group consisting of: SEQ ID NO: 3, SEQ ID NO: 4, SEQ ID NO: 5, SEQ ID NO: 6, and SEQ ID NO: 7.

In some embodiments of this aspect of the disclosure, the oligonucleotide can be admixed with a pharmaceutically acceptable diluent or carrier.

Another aspect of the disclosure encompasses embodiments of a method for modulating the expression of SMN2 in a cell expressing SMN2, the method comprising the step of administering an oligonucleotide of claim 1 to the cell or a population of cells.

In some embodiments of this aspect of the disclosure, the population of cells can be in a patient having a pathological condition associated with a reduction in motor neuron or neuromuscular junction numbers or with a disruption of motor neurons or neuromuscular junctions, and wherein the modulation of the expression of SMN2 can be a treatment for, or a prevention of, the pathological condition of the recipient patient.

Yet another aspect of the disclosure encompasses embodiments of a method of increasing a steady-state level of an mRNA encoding SMN2 in a subject in need thereof, the method comprising providing to the subject a therapeutically effective amount of an oligonucleotide of claim 1, wherein the oligonucleotide can promote the transcription of mRNA encoding SMN2, or inhibit degradation of an mRNA encoding SMN2 in cells of the subject in need thereof.

In some embodiments of this aspect of the disclosure, the subject can have a condition associated with a reduction in motor neuron or neuromuscular junction numbers, or a condition associated with disruption of motor neurons or neuromuscular junctions.

In some embodiments of this aspect of the disclosure, the subject ca have spinal muscular atrophy, has 5q spinal muscular atrophy, can be a geriatric subject, can have an acute muscle injury, or can have a chronic muscle injury.

In some embodiments of this aspect of the disclosure, the oligonucleotide can be co-administered with a splice-switching promoter, and wherein the oligonucleotide and the splice-switching promoter can be administered to the patient simultaneously or sequentially.

In some embodiments of this aspect of the disclosure, the splice-switching promoter can promote exon 7 inclusion in SMN2 mRNA.

In some embodiments of this aspect of the disclosure, the splice-switching promoter can be selected from the group consisting of: nusinersen, risdiplam, branaplam, or an oligonucleotide.

In some embodiments of this aspect of the disclosure, the oligonucleotide can comprise the nucleotide sequence of SEQ ID No: 14.

In some embodiments of this aspect of the disclosure, the splice-switching promoter can be nusinersen.

In some embodiments of this aspect of the disclosure, the oligonucleotide can be provided directly to the central nervous system of the subject.

In some embodiments of this aspect of the disclosure, the oligonucleotide can be provided directly to the cerebrospinal fluid of the subject.

In some embodiments of this aspect of the disclosure, the oligonucleotide can be provided to the subject by injection or by intrathecal injection.

In some embodiments of this aspect of the disclosure, the splice-switching promoter can be provided by injection.

In some embodiments of this aspect of the disclosure, the splice-switching promoter can be selected from risdiplam and branaplam, and is provided by oral administration.

In some embodiments of this aspect of the disclosure, the oligonucleotide can be provided in an amount of between about 5 mg to about 50 mg or between about 10 mg and about 15 mg.

### BRIEF DESCRIPTION OF FIGURES

FIGS. 1A-1C illustrate antisense oligonucleotides (ASO) targeting the 5′ end of SMN2 increase SMN protein levels in vitro.

FIG. 1A is illustrates ASOs designed for the SMN2 5′UTR. 2′-OMe antisense oligonucleotides were tiled in 2-nt increments along the beginning of the transcript, and as a control used an ASO directed toward the middle of the 5′UTR.

FIG. 1B illustrates SMN protein levels in SMN-deficient fibroblasts treated with 600 nM 5′UTR ASOs or a non-targeting control (NTC) oligonucleotide, where indicated.

FIG. 1C illustrates relative SMN protein levels normalized to alpha tubulin, and compared to levels in untransfected patient cells, represented by the dotted line. SMN levels from a carrier cell line were included as a reference, because this level of SMN expression is sufficient to be asymptomatic. n=3; * p<0.05 by t test in comparison to NTC ASO.

FIGS. 2A and 2B illustrate that an ASO targeting the 5′ end of SMN2 increases SMN mRNA levels in vitro.

FIG. 2A illustrates SMA patient fibroblasts transfected with 600 nM 2′-OMe 5′UTR ASO, splice-switching oligonudeotide (SSO), or non-targeting control oligonucleotide (NTC), as indicated. qRT-PCR measured total SMN mRNA levels. Expression was normalized to GAPDH, then compared to levels in un-transfected patient cells. n=3; * p<0.005 by t test in comparison to NTC ASO. Bars on the chart of FIG. 2A correspond to (from left to right) 5′UTR ASO; splice-switching ASO; non-targeting control ASO; untransfected; carrier.

FIG. 2B illustrates that 48 hours post-transfection with ASO, cells were treated with actinomycin D and collected in TRIzol at the specified time points. qRT-PCR measured total SMN mRNA. n=2; * p<0.05 by t test in comparison to NTC ASO at given time point.

FIGS. 3A and 3B illustrate that using a 5′UTR ASO in combination with a splice-switching ASO increases SMN protein levels more than use of the splice-switching oligonucleotide alone.

FIG. 3A illustrates SMA fibroblasts transfected with 600 nM of a 2′-OMe 5′UTR ASO, splice-switching oligonudeotide (SSO), a combination of the two (1200 nM), or a non-targeting control (NTC) oligonucleotide.

FIG. 3B illustrates SMN levels were normalized to alpha tubulin, and expression was compared to levels in untransfected patient cells. n=3; * p<0.05 by t test in comparison to combination treatment.

FIGS. 4A and 4B illustrate that a 5′UTR 2′-MOE ASO also increases SMN protein levels in fibroblasts.

An ASO with the same sequence as ASO #1 but with 2′-O-(2-methoxyethyl) (2′-MOE) modified bases was tested.

FIG. 4A illustrates SMN protein levels in SMA patient fibroblasts treated with 600 nM ASO, in either the 2′-OMe or 2′-MOE chemistry, as indicated.

FIG. 4B illustrates dose response with the 2′MOE ASO in SMA patient fibroblasts.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques can be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2nd Edition, Cold Spring Harbor Laboratory Press, 1989, which are hereby incorporated by reference for any purpose. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

### Abbreviations

Untranslated region, UTR; spinal muscular atrophy, SMA; survival motor neuron 1 gene, SMN1; survival motor neuron protein, SMN; small nuclear ribonucleoprotein, snRNP; survival motor neuron 2 gene, SMN2; survival motor neuron 2 protein, SMN2; amyotrophic lateral sclerosis, ALS; antisense oligonucleotide, ASO; non-targeting control, NTC; 2′-O-(2-methoxyethyl), 2′-MOE

### Definitions

The term (SMN1) as used herein refers to the telomeric copy of the gene encoding the SMN protein; the centromeric copy is termed SMN2. SMN1 and SMN2 are part of a 500 kb inverted duplication on chromosome 5q13. This duplicated region contains at least four genes and repetitive elements which make it prone to rearrangements and deletions. The repetitiveness and complexity of the sequence have also caused difficulty in determining the organization of this genomic region. SMN1 and SMN2 are nearly identical and encode the same protein. The critical sequence difference between the two is a single nucleotide in exon 7 which is thought to be an exon splice enhancer. It is thought that gene conversion events may involve the two genes, leading to varying copy numbers of each gene. Mutations in SMN1 are associated with spinal muscular atrophy. Mutations in SMN2 alone do not lead to disease, although mutations in both SMN1 and SMN2 result in embryonic death.

The term “oligonucleotide” as used herein is defined as it is generally understood by the skilled person as a molecule comprising two or more covalently linked nucleotides (or two or more covalently linked nucleosides). Such covalently bound nucleotides may also be referred to as oligomers. Such linked nucleotides may comprise natural nucleosides or modified nucleosides. Naturally occurring oligonucleotides comprise naturally occurring bases and furanosyl groups joined by native phosphodiester bonds. The term “oligonucleotide” as used herein also refers to moieties that have portions similar to naturally occurring oligonucleotides, but which have non-naturally occurring portions. Thus, oligonucleotides may have altered sugars, altered base moieties, or altered inter-sugar linkages. Among these are the phosphorothioate and other sulfur-containing species that are known in the art. Oligonucleotides are commonly made in the laboratory by solid-phase chemical synthesis followed by purification. The oligonucleotide of the disclosure is man-made, and is chemically synthesized, and is typically purified or isolated. The oligonucleotide of the disclosure may comprise one or more modified nucleosides or nucleotides.

The term “base sequence” as used herein refers to the sequential sequence of linked nucleobases and means the order of contiguous nucleobases independent of any sugar, linkage, or nucleobase modification. It may also be referred to as nucleobase sequence. The base sequence is typically displayed starting from 5′ end and finishing with 3′ end (5′-3′).

The term “antisense oligonucleotide” as used herein is defined as an oligonucleotide capable of hybridizing to a target nucleic acid, such as to the target region sequence disclosed herein, in particular to a contiguous sequence on a target nucleic acid in a sequence-specific manner. The antisense oligonucleotides are not essentially double stranded and are therefore not siRNAs or shRNAs. Advantageously, the antisense oligonucleotides of the present disclosure are single stranded. It is understood that single stranded oligonucleotides of the present disclosure can form hairpins or intermolecular duplex structures (duplex between two molecules of the same oligonucleotide), as long as the degree of intra or inter self-complementarity is less than 50% across of the full length of the oligonucleotide.

The term “contiguous nucleotide sequence” as used herein refers to the portion of the oligonucleotide which is complementary to or hybridizes to the target nucleic acid. Although this portion of the oligonucleotide is complementary to the target sequence, in some embodiments, not every nucleobase within the contiguous sequence need be complementary. Provided the contiguous nucleotide sequence can hybridize to the target region sequence then a mismatch, or in some embodiments more than 1 mismatch may exist. Advantageously, the contiguous nucleotide sequence is 100% complementary to the target region sequence. In some embodiments all the nucleotides of the oligonucleotide constitute the contiguous nucleotide sequence.

The term “nucleotide” as used herein refers to the building blocks of oligonucleotides and polynucleotides, and for the purposes of the present disclosure include both naturally occurring and non-naturally occurring nucleotides. In nature, nucleotides, such as DNA and RNA nucleotides comprise a five-carbon sugar (either ribose or deoxyribose), a nucleobase moiety (e.g. adenine, guanine, cytosine, uracil and thymine) and one or more phosphate groups joined in ester linkages to the sugar moiety.

The term “nucleoside” as used herein refers to a molecule having a purine or pyrimidine nucleobase covalently linked to a sugar moiety (e.g. ribose or deoxyribose sugar). Nucleosides include, but are not limited to, naturally occurring nucleosides (as found in DNA and RNA) and modified nucleosides. Nucleosides include adenosine, guanosine, cytidine, uridine and thymidine. A nucleoside consists of a nucleobase and a five-carbon sugar (either ribose or deoxyribose). Nucleosides are thus effectively nucleotides but without the phosphate groups attached to the sugar moiety.

The term “modified nucleoside” or “nucleoside modification” as used herein refers to nucleosides modified as compared to the equivalent DNA or RNA nucleoside by the introduction of one or more modifications to the sugar moiety or the (nucleo)base moiety. Such modifications are often at the 2′ position of the sugar moiety. In a preferred embodiment the modified nucleoside comprises a modified sugar moiety. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

The term “linkage” or “linking group” as used herein refers a group of atoms that link together two or more other groups of atoms.

The term “intemucleoside linkage” as used herein refers to a covalent linkage between adjacent nucleosides in an oligonucleotide.

The term “naturally occurring intemucleoside linkage” as used herein means a 3′ to 5′ phosphodiester linkage.

The term “modified intemucleoside linkage” as used herein, means any intemucleoside linkage other than a naturally occurring intemucleoside linkage. The term “intemucleoside phosphorus linking group” as used herein means a phosphorus linking group that directly links two nucleosides The oligonucleotides of the disclosure may comprise modified inter-nucleoside linkages.

In some embodiments of the disclosure, the modified inter-nucleoside linkage increases the nuclease resistance of the oligonucleotide compared to a phosphodiester linkage. For naturally-occurring oligonucleotides, the inter-nucleoside linkage includes phosphate groups creating a phosphodiester bond between adjacent nucleosides. Modified inter-nucleoside linkages are particularly useful in stabilizing oligonucleotides for in vivo use and may serve to protect against nuclease cleavage at regions of DNA or RNA nucleosides in the oligonucleotide of the disclosure.

In some embodiments of the disclosure, the oligonucleotide comprises one or more inter-nucleoside linkages modified from the natural phosphodiester, such one or more modified inter-nucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Inter-nucleoside linkages which are capable of enhancing the nuclease resistance of an oligonucleotide are referred to as nuclease resistant inter-nucleoside linkages. In some embodiments at least 50% of the inter-nucleoside linkages in the oligonucleotide, or contiguous nucleotide sequence thereof, are modified, such as at least 60%, such as at least 70%, such as at least 80 or such as at least 90% of the inter-nucleoside linkages in the oligonucleotide, or contiguous nucleotide sequence thereof, are nuclease resistant inter-nucleoside linkages. In some embodiments all of the inter-nucleoside linkages of the oligonucleotide, or contiguous nucleotide sequence thereof, are nuclease resistant inter-nucleoside linkages.

The term “phosphorus linking group” as used herein refers a linking group comprising a phosphorus atom. Phosphorus linking groups are well known in the art and include without limitation, phosphodiester, phosphorothioate, phosphorodithioate, phosphonate, phosphoramidate, phosphorothioamidate, thionoalkylphosphonate, phosphotriesters, thionoalkylphosphotriester and boranophosphate.

The term “intemucleoside phosphorus linking group” as used herein refers to a phosphorus linking group that directly links two nucleosides. A preferred modified inter-nucleoside linkage is phosphorothioate. Such linkages are particularly useful due to nuclease resistance, beneficial pharmacokinetics and ease of manufacture.

In some embodiments of the disclosure, at least 50% of the inter-nucleoside linkages in the oligonucleotide, or contiguous nucleotide sequence thereof, are phosphorothioate, such as at least 60%, such as at least 70%, such as at least 80% or such as at least 90% of the inter-nucleoside linkages in the oligonucleotide, or contiguous nucleotide sequence thereof, are phosphorothioate. In some embodiments all of the inter-nucleoside linkages of the oligonucleotide, or contiguous nucleotide sequence thereof, are phosphorothioate.

Advantageously, all the inter-nucleoside linkages in the contiguous nucleotide sequence of the oligonucleotide are phosphorothioate linkages. Further, all the inter-nucleoside linkages in the oligonucleotide sequence may be phosphorothioate linkages.

The term “nucleobase” as used herein includes the purine (e.g. adenine and guanine) and pyrimidine (e.g. cytosine, thymine and uracil) moiety present in nucleosides and nucleotides which form hydrogen bonds in nucleic acid hybridization. In the context of the present disclosure, the term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 p. 2055, and Bergstrom (2009) Current Protocols in Nucleic Acid Chemistry Suppl. 37 1.4.1. Modified purine or pyrimidine nucleobases are known in the art. The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine.

The term “modified oligonucleotide” as used herein refers to an oligonucleotide comprising one or more sugar-modified nucleosides and/or modified inter-nucleoside linkages.

The term “complementarity” as used herein refers to the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al., Accounts of Chemical Research 45: 2055, 2012, and Bergstrom. Current Protocols in Nucleic Acid Chemistry Suppl. 37 1.4.1, 2009).

The term “% complementary” as used herein refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target region sequence). The percentage is calculated by counting the number of aligned bases that form pairs between the two sequences (when aligned with the target region sequence 5′-3′ and the oligonucleotide sequence from 3′-5′), dividing by the total number of nucleotides in the oligonucleotide and multiplying by 100. In such a comparison the % identity is only measured across the length of the shorter of the two sequences. In such a comparison a nucleobase/nucleotide which does not align (form a base pair) is termed a mismatch. Preferably, insertions and deletions are not allowed in the calculation of % complementarity of a contiguous nucleotide sequence. The term “fully complementary”, refers to 100% complementarity.

The term “sequence identity” as used herein with respect to a nucleotide sequence refers to the percentage of nucleotide residues in a candidate target sequence that are identical with the nucleotide residues in a subject nucleotide sequence, after aligning the sequences and allowing for gaps, if necessary, to achieve the maximum percent sequence identity, and not considering any conservative substitutions as part of the sequence identity. Alignment for purposes of determining percent nucleotide sequence identity can be achieved in various ways that are within the skill of one in the art, for instance, using publicly available computer software such as BLAST, BLAST-2, ClustalW2, ALIGN or MEGALIGN™ (DNASTAR) software. Those skilled in the art can determine appropriate parameters for measuring alignment, including any algorithms needed to achieve maximal alignment over the full length of the sequences being compared. For example, sequence identity of two or more nucleotide sequences can be determined by using the algorithm of Karlin and Altschul, BLAST (Basic Local Alignment Search Tool) (Proc. Natl. Acad. Sci. USA 872264-2268, 1990; Proc Natl Acad Sci USA 90: 5873, 1993). Based on the algorithm of BLAST, programs called BLASTN and BLASTX have been developed (Altschul et al., J. Mol. Biol. 215: 403, 1990). If BLASTN is used for nucleotide sequence analysis, parameters may be set to, for example, score=100 and word length=12. If BLAST and Gapped BLAST programs are used, default parameters in each program may be used. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

The term “hybridization” as used herein refers to the pairing of complementary nucleic acid strands, e.g. oligomeric compounds (e.g., an antisense compound and its target nucleic acid), thereby forming a duplex. While not limited to a particular mechanism, the most common mechanism of pairing involves hydrogen bonding, which may be Watson-Crick, Hoogsteen or reversed Hoogsteen hydrogen bonding, between complementary nucleobases.

The term “specifically hybridizes” as used herein refers to the ability of an oligomeric compound to hybridize to one nucleic acid site with greater affinity than it hybridizes to another nucleic acid site. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny & Lacroi. Oligonucleotides 13:515-537, 2003). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by (SantaLucia, (1998) Proc. Natl. Acad. Sci. U.S.A. 95: 1460-1465 using appropriately derived thermodynamic parameters described by Sugimoto et al., (1995) Biochemistry 34:11211-11216 and McTigue et al., (2004) Biochemistry 43: 5388-5405. To have the possibility of modulating its intended nucleic acid target by hybridization, oligonucleotides of the present disclosure hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

The term “target region” (also referred to herein as “target region sequence” or “target region nucleic acid”) as used herein refers to a region of nucleic acid located at and around the 5′UTR of SMN2 gene (including pre-mRNA and mRNA) that comprises the nucleobase sequence which is complementary to the oligonucleotide of the disclosure. The target region sequence consists of a region on the target nucleic acid which is complementary to the contiguous nucleotide sequence of the oligonucleotide of the disclosure. The target region may therefore be referred to as an SMN2 target nucleic acid. For information, the locus of the target region is chr5:70049572-70049604 in UCSC hg38.

In embodiments of the disclosure, an advantageous target region is all or a part of the sequence disclosed in SEQ ID NO: 1 or naturally occurring variants thereof.

There are a few minor allele single nucleotide polymorphism variants within the target region sequence (identified through Ensembl, TOPmed population genetics). Table 1 provides examples of major and minor allele frequencies for bases at 6 locations in the target region sequence, wherein N (number) refers to the base (where N can be A,C,G, or T) and “number” refers to the position in SEQ ID NO: 1; so, e.g. T (10) refers to the thymine at position 10 in SEQ ID NO: 1. The allele frequency for each recited base is then provided.

TABLE 1
Naturally-occurring SNP frequencies in SEQ ID NO: 1
identified through Ensembl, TOPmed population genetics.
CCACAAATGTGGGAGGGCGATAACCACTCGTAG
(SEQ ID NO: 1)

| SNP | Allele frequency | | |
|---|---|---|---|
| T (10) | T: 0.999960181 | C: 2.38910e−05 | G: 1.59280e−05 |
| G (12) | G: 0.996 | A: 0.004 | |
| T (21) | T: 0.999992036 | A: 7.96400e−06 | |
| A (26) | A: 0.999984072 | C: 1.59280e−05 | |
| T (28) | T: 0.999984072 | C: 1.59280e−05 | |
| G (30) | G: 0.999976108 | A: 1.59280e−05 | C: 7.96400e−06 |

For in vivo or in vitro application, the oligonucleotides of the disclosure are typically capable of enhancing the expression of the SMN2 target nucleic acid in a cell tat is expressing the SMN2 target nucleic acid. The contiguous sequence of nucleobases of the oligonucleotide of the disclosure is typically complementary to the part of the SMN2 target nucleic acid, as measured across the length of the oligonucleotide, optionally with no more than one or two mismatches. The target nucleic acid is a messenger RNA, such as a mature mRNA or a pre-mRNA which encodes mammalian SMN protein, such as human SMN2, e.g. the human SMN2 pre-mRNA sequence. The reference sequence of one SMN2 transcript is NM_017411. I.

It will be understood that target RNA sequences have uracil (U) bases in place of the thymidine bases (T).

In some embodiments of the disclosure, the target region sequence is longer than the complementary sequence of a single oligonucleotide, and may, for example represent a preferred region of the target nucleic acid which may be targeted by several oligonucleotides of the disclosure.

The oligonucleotides of the disclosure comprise a contiguous nucleotide sequence which is complementary to or hybridizes to the target region nucleic acid, such as a sub-sequence of the target nucleic acid, such as a target sequence described herein.

The term a “target cell” as used herein refers to a cell which is expressing the target region nucleic acid and SMN2 gene. In some embodiments, the target cell may be in vivo. In other embodiments the cell may be an isolated cell or population of cells, or a cultivated cell or population of cells under in vitro conditions well-known to those in the art In embodiments of the disclosure the target cell can be a mammalian cell such as a rodent cel, such as a mouse cell or a rat cell, or a primate cell such as a monkey cell or a human cell.

The term “expression” as used herein refers to the process by which a gene ultimately results in a protein. Expression includes, but is not limited to, transcription, post-transcriptional modification (e.g., splicing, polyadenlyation, addition of 5′-cap), and translation.

The term “increase in expression” as used herein is to be understood as an overall term for an agent's ability to increase (induce) the amount of SMN protein or SMN2 mRNA when compared to the amount of SMN2 or SMN2 mRNA prior to administration of an agent such as an oligonucleotide). Advantageously, the oligonucleotides of the disclosure are capable of increasing the expression of SMN2 mRNA in a cell which is expressing SMN2 mRNA. Advantageously, the oligonucleotides of the disclosure are capable of increasing the expression of SMN1 mRNA in a cell which is expressing SMN1 mRNA (e.g. in non-SMA patients that have a functional SMN1 gene).

Sugar-Modified Nucleotides:

The oligonucleotides of the disclosure may comprise one or more nucleosides that have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituted sugar moieties include but are not limited to furanosyls comprising substituents at the 2′-position, the 3′-position, the 5′-position and/or the 4′-position. Certain substituted sugar moieties are bicyclic sugar moieties.

The term “2′ sugar modified nucleosides” as used herein refers to a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

The term 2′ sugar substituted nucleoside” as used herein refers to a nucleoside comprising a substituent at the 2′-position other than H or OH. Unless otherwise indicated, a 2′-substituted nucleoside is not a bicyclic nucleoside.

Numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples, not intended to be limiting, of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. Further examples are provided by Freier & Altmann (1997) Nucl. Acid Res. 25: 4429-4443, Uhlmann (2000) Curr. Opinion Drug Development 3: 293-213, and Deleavey & Damha (2012) Chemistry and Biology 19: 937, each of which is incorporated herein by reference in its entirety.

Advantageous 2′ modifications for use in the embodiments of the disclosure include 2′-O-MOE and 2′-O-Me. As used herein, “2′-O-methoxyethyl” (also 2′-MOE and 2′-O(CH2)2-OCH3) refers to an O-methoxyethyl modification of the 2′ position of a furosyl ring. A 2′-O-methoxyethyl modified sugar is a modified sugar. As used herein, “2′-O-methoxyethyl nucleotide” means a nucleotide comprising a 2′-O-methoxyethyl modified sugar moiety.

The terms “locked nucleic acid nucleoside” or “LNA” as used herein refers to a nucleoside comprising a bicyclic sugar moiety comprising a 4′-CH₂—O-2′ bridge, which restricts or locks the conformation of the ribose ring. Such nucleosides are also referred to in the art as bridged nucleic acid or bicyclic nucleic acid (BNA). As used herein, “bicyclic nucleoside” or “BNA” means a nucleoside comprising a bicyclic sugar moiety.

Non-limiting examples of LNA nucleosides for use in the embodiments of the disclosure are given in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic &Med.Chem. Left. 12, 73-76, Seth et al. J. Org. Chem. 2010, Vol 75(5) pp. 1569-1581, and Mitsuoka et al., Nucl. Acids Res. 2009, 37(4), 1225-1238, and Wan & Seth, J. Med. Chem. 2016, 59, 9645-9667.

Some advantageous LNA nucleosides are beta-D-oxy-LNA, 6′-methyl-beta-D-oxy LNA such as (S)-6′-methyl-beta-D-oxy-LNA (ScET) and ENA.

The term “constrained ethyl nucleoside” or “cEt” as used herein refers to a nucleoside comprising a bicyclic sugar moiety comprising a 4′-CH(CH₃)—O-2′ bridge.

The terms “peptide nucleic acid” or “PNA” as used herein refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard Solid phase pep tide synthesis protocols as described in Hyrup et al., (1996) Bioorganic &Med. Chem. 4(1):5-23; and Perry-O'Keefe et al., (1996) Proc. Natl. Acad. Sci. USA 93: 14670-675.

### Morpholino Oligomer

The term “morpholino oligomer” of the present disclosure refers to an antisense oligomer according to the present disclosure, whose constituent unit is a group represented by the following general formula:

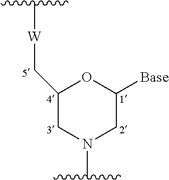

(wherein Base is the nucleobase; and W represents a group represented by any of the following formulae:

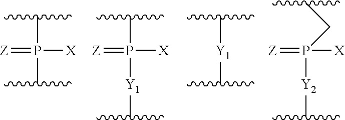

wherein X represents —CH₂R¹, —O—CH₂R¹, —S—CH₂R¹, —NR²R³ or F; R¹ represents H or alkyl; R² and R³ are each independently H, alkyl, cycloalkyl or aryl; Y₁ represents 0, S, CH₂ or NR¹; Y₂ represents O, S or NR¹; and Z represents 0 or S)).

The morpholino oligomer is preferably an oligomer whose constituent unit is a group represented by the following formula (i.e., a phosphorodiamidate morpholino oligomer (hereinafter referred to as “PMO”)):

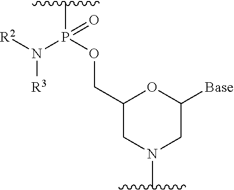

(wherein Base, R² and R³ are the same as defined above).

For example, the morpholino oligomer may be prepared in accordance with WO1991/009033 or WO2009/064471.

The term “pharmaceutical composition” as used herein refers to a mixture of substances advantageous for administering to an individual. For example, a pharmaceutical composition can comprise one or more active agents and a sterile aqueous solution.

The term “pharmaceutically acceptable carrier” as used herein refers to a medium or additive that does not interfere with the structure or function of the oligonucleotide. Certain, of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection or infusion. For example, a pharmaceutically acceptable carrier can be a sterile aqueous solution.

The carrier may serve to promote the delivery of the oligonucleotide to particular tissue. Such a carrier is not limited in any way as long as it is pharmaceutically acceptable. Advantageous examples include cationic carriers (e.g., cationic liposomes, cationic polymers) or viral envelope-based carriers. Examples of cationic liposomes include liposomes formed from 2-O-(2-diethylaminoethyl)carbamoyl-1,3-O-dioleoyl glycerol and a phospholipid as essential constituent members (hereinafter referred to as “liposome A”), Oligofectamine® (Invitrogen), Lipofectin® (Invitrogen), Lipofectamine® (Invitrogen), Lipofectamine 2000® (Invitrogen), DMRIE-C® (Invitrogen), GeneSilencer® (Gene Therapy Systems), TransMessenger® (QIAGEN), TransiT TKO® (Mirus) and Nucleofector II (Lonza). Among them, preferred is liposome A. Examples of cationic polymers include JetSI® (Qbiogene) and Jet-PEI® (polyethyleneimine, Qbiogene).

For more details, reference may be made to U.S. Pat. Nos. 4,235,871 and 4,737,323, WO96/14057, and to New RRC, Liposomes: A practical approach, IRL Press, Oxford (1990) pages 33-104.

The term “pharmaceutically acceptable diluent” as used herein refers to a diluting agent advantageous for pharmaceutical use. Phosphate buffered saline is an advantageous example. The term “pharmaceutically acceptable salts” as used herein refers tocphysiologically and pharmaceutically acceptable salts of antisense oligonucleotide compounds, i.e., salts that retain the desired biological activity of the parent oligonucleotide and do not impart undesired toxicological effects thereto.

The term “treatment” as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Treatment may be taken as encompassing any improvement in a symptom or reduction in pathology associated with a disease that occurs on provision of an agent of the disclosure. In particular, treatment may give rise to a clinically significant improvement in symptoms or reduction in pathology on provision of an agent of the disclosure (whether in monotherapy or combination therapy). References of the disclosure to therapeutically effective amounts should be interpreted in a manner consistent with these definitions.

When used in a method of the disclosure, an agent of the disclosure is provided in an effective (or therapeutically effective, in a method of treatment) amount. This effective, or therapeutically effective, amount is an amount of an agent of the disclosure that alone, or together with further doses, produces a desired response.

In the context of a method of the disclosure, an effective amount can be an amount capable of bringing about a desired promotion of the transcription of SMN2 in a subject or an effective amount is an amount capable of bringing about a desired inhibition of degradation of SMN2 mRNA in a subject.

In some embodiments of the disclosure, an effective amount can an amount capable of bringing about a desired increase in steady-state levels of SMN2 mRNA in a subject.

In some embodiments of the disclosure, a therapeutically effective amount is an amount capable of bringing about a desired improvement in a symptom, or reduction in a pathology, associated with SMA.

Advantageously, an effective or therapeutically effective amount may be an amount that is capable of increasing SMN2 gene expression, and/or increasing SMN2 mRNA production, and/or increasing production of SMN protein.

A therapeutically effective amount of an agent of the disclosure (or of another therapeutic agent, such as a substance that promotes splice-switching, for use in a combination therapy) may be provided by means of a single dose. Alternatively, the required therapeutically effective amount may be provided by a number of doses that cumulatively provide the therapeutically effective amount.

Merely by way of example, the agent of the disclosure may be provided in a therapeutically effective amount of between about 5 mg and about 50 mg. Advantageously, the agent is provided in a therapeutically effective amount of between about 10 mg and about 15 mg. Advantageous doses of agents of the disclosure, that may be employed in the pharmaceutical compositions or methods of the disclosure, are considered elsewhere in the specification.

Treatment with the Methods of the Disclosure and Conditions to be Treated

The methods of the disclosure may be of use in the prevention or treatment of diseases or conditions mediated by or associated with decreased SMN protein production. In advantageous embodiments, the methods of the disclosure may be of use in the prevention or treatment of diseases or conditions that will benefit from increased SMN2 mRNA production or full-length SMN protein production.

They may be of particular benefit in the treatment of a subject who has a disease or condition associated with a reduction in motor neuron or neuromuscular junction numbers, or a disease or condition associated with disruption of motor neurons or neuromuscular junctions. For example, such methods may be used in the treatment of a subject who has a disease or condition associated with a reduction in motor neuron numbers. Similarly, such methods may be used in the treatment of a subject who has a disease or condition associated with a reduction in neuromuscular junction numbers. Alternatively, or additionally, methods in accordance with these aspects may be used in the treatment of a subject who has a disease or condition associated with disruption of motor neurons. Such methods may be used in the treatment of a subject who has a disease or condition associated with disruption of neuromuscular junctions.

The skilled person will recognize many such diseases or conditions, including, but not limited to SMA and ALS. Advantageously a subject may have a disease selected from the group consisting of: SMA and ALS. It is a particularly preferred embodiment, the methods of the disclosure can be used in the treatment of a subject with spinal muscular atrophy.

Other examples of condition associated with a reduction in motor neuron or neuromuscular junction numbers or a disruption of such neurons or junctions include aging and trauma of the muscles. Thus, subjects able to benefit from treatment by methods in accordance with the methods of the disclosure may also include those selected from the group consisting of: a geriatric subject; a subject with acute muscle injury; and a subject with chronic muscle injury.

The term “SMA” as used herein, refers to spinal muscular atrophy, a human autosomal recessive disease that is often characterized by under-expression of SMN protein in affected individuals. Methods of the disclosure are directed to the treatment of SMA. Methods of the disclosure may be used in the treatment of SMA.

A number of different types of SMA have been identified. These include SMA1 (infantile onset SMA, also known as Werdnig-Hoffmann disease), SMA2 (intermediate onset SMA, also known as Dubowitz disease), SMA3 (juvenile onset SMA, also known as Kugelberg-Welander disease), and SMA4 (adult onset SMA). The agents and methods of the disclosure can be advantageous in the treatment of a subject with SMA1, with SMA2, with SMA3, or with SMA4.

SMA may also be characterized with reference to the location of the mutation associated with the disease. The most common form of SMA, according to this classification, is 5q SMA, in which the relevant mutation is located on the “q” region of chromosome 5. The agents and methods of the disclosure can be useful in the treatment of a subject with 5q SMA. Advantageously the agents or methods of the disclosure may be used in the treatment of a subject that has 5q spinal muscular atrophy.

The term “Amyotrophic lateral Sclerosis (ALS)” (Lou Gehrig's disease”) is another neurological disease characterized by low levels of SMN protein (Veldink et al. 2005 Neurology 65(6):820-5).

### Discussion

The present disclosure is based on the discovery that antisense oligonucleotides which bind to all or part of the 5′UTR of SMN2 induce the cell to produce more SMN2 mRNA and thus more SMN protein.

The 5′UTR of SMN1 and SMN2 is the same, so in non-SMA cells the agent of the disclosure (e.g. antisense oligonucleotide) will operate by binding to (and blocking) both SMN1 and SMN2 transcripts. In SMA patients, the increase in SMN protein generated is mostly through blocking of 5′UTR of SMN2, as SMN1 gene is mutated and little to no SMN protein is produced from SMN1. When referring to blocking of 5′UTR of SMN2 herein, it will thus be appreciated that this can also mean blocking of 5′ UTR of SMN1. Thus, an SMN2 5′ UTR blocking agent, as referred to herein, is one that binds to the target region (which as noted above is the same for SMN1 and SMN2).

The disclosure provides an agent capable of binding to all or part of a target region that comprises the 5′ UTR of SMN2. The disclosure is directed to effective use of SMN2 5′ UTR blocking agents, in particular, blocking oligonucleotide molecules (e.g., modified antisense oligoribonucleotides) capable of binding to the 5′ UTR SMN2 target region sequence. Such binding may prevent ribosome binding or preventing the binding of proteins or non-coding RNAs that promote RNA degradation. Advantageously, the SMN2 is human SMN2. The target region has the nucleic acid sequence disclosed in SEQ ID NO: 1. The complement to this target region I disclosed in SEQ ID NO: 2. Treatment of cells derived from SMA patients with the oligonucleotide reagent compositions of the present disclosure increases the production of SMN2 mRNA and, in particular, the amount of full-length SMN protein. Cells into which the agent has been delivered and wherein the agent has bound to the target region produce more SMN2 mRNA and/or SMN protein. The ability to increase the amount of SMN protein is particularly advantageous in patients with mutations in SMN1, who therefore rely solely on SMN2 for SMN protein production. Due to the C to T transition in exon 7 of SMN2, exon skipping occurs about 80-90% of the time so that only 10-20% functional protein is produced. By increasing the amount of SMN2 mRNA in accordance with the disclosure, more functional SMN protein is generated.

Furthermore, a relatively recent therapeutic approach for patients with mutations in SMN1 is to treat them with an agent that promotes exon 7 inclusion in the SMN2 transcript. Increasing the total pool of mRNA transcript in accordance with the present disclosure, would likely provide more transcript for such exon skipping agents to act upon, with the result that more functional SMN protein can be produced. The agent of the disclosure can therefore be used as monotherapy or as part of a combined therapy approach for the treatment of spinal muscular atrophy (SMA).

Increasing SMN levels in ALS iPSC-derived motor neurons was shown to increase motor neuron survival (Rodriguez-Muela et al. Cell Reports 18:1484-1498, 2017). Accordingly, the agents of the disclosure may also be useful in the treatment of amyotrophic lateral Sclerosis (ALS), another neurological disease characterized by low levels of SMN protein (see also Veldink et al. Neurology 65(6):820-5, 2005).

The methods of the disclosure can be carried out using any agent capable of binding to the target region. Examples of advantageous agents include small molecules, DNA binding proteins, peptide aptamers and nucleic acid molecules, such as oligonucleotides. Antisense oligonucleotides are particularly useful in the practice of the disclosure.

According there is provided an agent capable of binding to a 5′UTR SMN2 target region sequence having the sequence as disclosed in SEQ ID NO: 1. Examples of advantageous agents include small molecules, DNA binding proteins, peptide aptamers and nucleic acid molecules, such as oligonucleotides. Antisense oligonucleotides are particularly useful in the practice of the disclosure. Following binding of the agent to the target region sequence in a cell, the cell produces more SMN2 mRNA and/or SMN protein compared to when the agent is not present. Particularly advantageous agents are oligonucleotides that can hybridize to the some or all of the target region sequence.

Further provided an oligonucleotide, 17 to 35 nucleotides in length, wherein said oligonucleotide comprises a base sequence that is complementary to at least 10 consecutive bases of a target region sequence having the sequence as disclosed in SEQ ID NO: 1; or a pharmaceutically acceptable salt thereof.

Provided an oligonucleotide, 17 to 35 nucleotides in length, wherein said oligonucleotide comprises a base sequence that is complementary across its' length to a target region sequence having the sequence as disclosed in SEQ ID NO: 1, aside from the presence of up to 5 nucleobase substitutions; or a pharmaceutically acceptable salt thereof.

The oligonucleotide can be of any length, such as from 10-60 nucleotides in length, but typically will be 17 to 35 nucleotides in length. Advantageously the oligonucleotide is isolated.

SEQ ID NO:1 provides the sequence of a portion of the 5′UTR of SMN2 and a few additional bases upstream (It is the first 33 bases of the SMN2 transcript). In the context of this disclosure this region is referred to as the “target region” and this sequence is referred to as the “target region sequence”. SEQ ID NO: 2 provides the complement sequence to SEQ ID NO: 1.

The oligonucleotide of the disclosure is capable of hybridizing to all or part of the target region nucleic acid in a cell. Following such hybidization/binding, the cell is capable of increased expression of SMN2 mRNA and/or decreased mRNA degradation and/or increased total SMN protein expression

The present disclosure is also directed to an isolated oligonucleotide sequence comprising the sequence 5′-GTTATCGCCCTCCCACATTTGTGG-3′ (SEQ ID NO: 3; ASO #1), or as sequence with at least 80%, such as at least 90%, sequence identity thereto, to an isolated oligonucleotide sequence comprising the sequence 5′-TGGTTATCGCCCTCCCACATTGT-3′ (SEQ ID NO: 4; ASO #2) or as sequence with at least 80%, such as at least 90%, sequence identity thereto, to an isolated oligonucleotide sequence comprising the sequence 5′-AGTGGTTATCGCCCTCCCACATTT-3′ (SEQ ID NO: 5; ASO #3) or as sequence with at least 80%, such as at least 90%, sequence identity thereto.

An isolated oligonucleotide sequence can comprise the sequence 5′-3′ (SEQ ID NO: 6; ASO #4) or as sequence with at least 80%, such as at least 90%, sequence identity thereto or an isolated oligonucleotide sequence comprising the sequence SEQ ID NO: 7 (ASO #5) or as a sequence with at least, such as at least 90%, sequence identity thereto.

The disclosure further provides a pharmaceutical composition comprising the agent or oligonucleotide of the disclosure and a pharmaceutically acceptable diluent or carrier. The pharmaceutical composition may be in lyophilized form.

Also provided by the disclosure is an in vivo or in vitro method for modulating SMN2 expression in a target cell which is expressing SMN2, the method comprising administering an agent, such as an oligonucleotide, or pharmaceutical composition of the disclosure in an effective amount to said cell.

A method of the disclosure allows for promoting the transcription of SMN2 in a subject in need thereof, the method comprising providing to the subject an effective amount of an agent of the disclosure.

The disclosure further provides a method of inhibiting degradation of SMN2 mRNA in a subject in need thereof, the method comprising providing to the subject an effective amount of an agent of the disclosure.

Also provided a method of therapeutically increasing steady-state levels of SMN2 mRNA in a subject in need thereof, the method comprising providing to the subject an effective amount of an agent of the disclosure.

The disclosure provides for a method for treating or preventing a disease comprising administering a therapeutically or prophylactically effective amount of an agent of the disclosure or a pharmaceutical composition of the disclosure to a subject suffering from or susceptible to the disease and a method of treating spinal muscular atrophy in a subject in need thereof, the method comprising providing to the subject a therapeutically effective amount of an agent of the disclosure. The latter method may optionally employ further therapeutic agents, giving rise to combination therapies as considered in more detail elsewhere in the specification.

The disclosure also provides a method of treating spinal muscular atrophy in a subject in need thereof, the method comprising providing to the subject a therapeutically effective amount of an agent of the disclosure, and a therapeutically effective amount of an agent that promotes splice-switching.

The disclosure relates to agents in particular, but not limited to, antisense oligonucleotides, capable of binding to 5′UTR of SMN2 and effecting an increase in SMN2 mRNA and protein. The oligonucleotides of the disclosure targeting 5′UTR of SMN2 are capable of hybridizing to and increasing the expression of a SMN2 target nucleic acid in a cell which is expressing the SMN2 target nucleic acid. The SMN2 target nucleic acid may be a mammalian SMN2 mRNA or pre-mRNA, such as a human SMN2 mRNA or pre-mRNA, for example a pre-mRNA or mRNA originating from the Homo sapiens SMN2, see the following database entries:

One aspect of the disclosure encompasses embodiments of an agent capable of binding to a 5′UTR SMN2 target region sequence having the sequence as disclosed in SEQ ID NO: 1. Examples of advantageous agents include small molecules, DNA binding proteins, peptide aptamers and nucleic acid molecules, such as oligonucleotides. Antisense oligonucleotides are particularly useful in the practice of the disclosure. Following binding of the agent to the target region sequence in a cell, the cell produces more SMN2 mRNA and/or SMN protein compared to when the agent is not present. Particularly advantageous agents are oligonucleotides that can hybridize to the some or all of the target region sequence.

According to another aspect of the disclosure, there is provided an oligonucleotide, 17 to 35 nucleotides in length, wherein said oligonucleotide comprises a base sequence that is complementary to at least 10 consecutive bases of a target region sequence having the sequence as disclosed in SEQ ID NO: 1; or a pharmaceutically acceptable salt thereof. The oligonucleotide can be of any length, such as from 10-60 nucleotides in length, 15 to 45, 17 to 35, 15 to 24, 15 to 26, and 20 to 40 in length. One advantageous length is 17 to 35 nucleotides in length. Advantageously the oligonucleotide is isolated.

The oligonucleotide of the present disclosure may have a length of 10 to 60 nucleotides, such as: 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 10 to 22, 11 to 22, 12 to 22, 13 to 22, 14 to 22, 15 to 22, 16 to 22, 17 to 22, 18 to 22, 19 to 22, 20 to 22, 21 to 22, 10 to 21, 11 to 21, 12 to 21, 13 to 21, 14 to 21, 15 to 21, 16 to 21, 17 to 21, 18 to 21, 19 to 21, 20 to 21, 10 to 20, 11 to 20, 12 to 20, 13 to 20, 14 to 20, 15 to 20, 16 to 20, 17 to 20, 18 to 20, 19 to 20, 10 to 19, 11 to 19, 12 to 19, 13 to 19, 14 to 19, 15 to 19, 16 to 19, 17 to 19, 18 to 19, 10 to 18, 11 to 18, 12 to 18, 13 to 18, 14 to 18, 15 to 18, 16 to 18, 17 to 18, 10 to 17, 11 to 17, 12 to 17, 13 to 17, 14 to 17, 15 to 17, 16 to 17, 10 to 16, 11 to 16, 12 to 16, 13 to 16, 14 to 16, 15 to 16, 10 to 15, 11 to 15, 12 to 15, 13 to 15 and 14 to 15 nucleotides from its 5′ end to the 3′ end.

The antisense oligonucleotide of the present disclosure may have a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides from its 5′ end to the 3′ end (hereinafter referred to as “exemplary length of the antisense oligomer of the present disclosure”). Particularly advantageous ranges for the length of the oligomer of the disclosure include: 15 to 45, 17 to 35, 15 to 24, 15 to 26, and 20 to 40 nucleotides from its 5′ end to the 3′ end.

In particular embodiments, the base sequence is complementary to at least 11, 12, 13, 14, 15, 16, 17, 18, 19, 20, 21, 22, 23, 24, 25, 26, 27, 28, 29, 30, 31, 32 or 33 consecutive bases of the target region sequence as disclosed in SEQ ID NO: 1. In a particular embodiment, the base sequence is 100% complementary to the target sequence. In a particular embodiment, the base sequence is complementary to at least 12-20 consecutive bases of the sequence disclosed in SEQ ID NO: 1. In a particular embodiment, the base sequence is fully complementary to all or part of SEQ ID No: 1.

In particular embodiments, the oligonucleotide of the disclosure can comprise a contiguous nucleotide sequence that is complementary to a sequence present in the target region sequence. The contiguous nucleotide sequence comprises at least 10 contiguous nucleotides, such as 10, 11, 12, 13, 14, 15, 16, 17, 18, 19, 20, 21, 22, 23, 24, 25, 26, 27, 28, 29, 30, 31, 32 or 33, such as from 10-33, such as from 14-30, such as from 18-26, contiguous nucleotides.

SEQ ID Nos: 8-12 Represent the Target Sequence that AON # s 1-5 (SEQ ID Nos: 3-7) Bind to.

In some embodiments, the contiguous nucleotide sequence is fully complementary to SEQ ID NO 8.

In some embodiments, the contiguous nucleotide sequence is fully complementary to SEQ ID NO 9.

In some embodiments, the contiguous nucleotide sequence is fully complementary to SEQ ID NO 10.

In some embodiments, the contiguous nucleotide sequence is fully complementary to SEQ ID NO 11.

In some embodiments, the contiguous nucleotide sequence is fully complementary to SEQ ID NO 12.

An aspect of the present disclosure relates to an antisense oligonucleotide, such as a 2′-MOE or 2′-OMe modified antisense oligonucleotide that comprises a contiguous nucleotide sequence of 17 to 35 nucleotides in length with at least 90% complementarity, such as is fully complementary to SEQ ID NO 1.

In some embodiments, the oligonucleotide comprises a contiguous sequence of 10-33 nucleotides, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid sequence as present in SEQ ID No: 1.

In some embodiments, the oligonucleotide of the disclosure comprises a contiguous nucleotides sequence of 12-24, such as 13, 14, 15, 16, 17, 18, 19, 20, 21, 22, or 23 contiguous nucleotides in length, wherein the contiguous nucleotide sequence is fully complementary to SEQ ID NO 1.

In some embodiments of the present disclosure the oligonucleotide of the disclosure is at least 60% complementary to the target region sequence, such as at least 80% or at least 90%, complementary to the target region sequence. 100% complementarity of the oligonucleotide of the present disclosure to a target region sequence is particularly advantageous.

In other embodiments, the base sequence is a sequence that has at least 80%, such as at least 85%, 90% or 95%, sequence identity to a sequence that is complementary to the target region sequence.

According to a variation of the second aspect of the disclosure there is provided an oligonucleotide, 17 to 35 nucleotides in length, wherein said oligonucleotide comprises a base sequence that is complementary across its' length to a target region sequence having the sequence as disclosed in SEQ ID NO: 1, aside from the presence of up to 5 nucleobase substitutions; or a pharmaceutically acceptable salt thereof. The term “up to 5 nucleobase substitutions” means that there may be 1, 2, 3, 4 or 5 nucleobases in the oligonucleotide that do not complement with the target region sequence.

Advantageously, in some embodiments all of the inter-nucleoside linkages between the nucleosides of the contiguous nucleotide sequence are phosphorothioate inter-nucleoside linkages.

The disclosure provides antisense oligonucleotides such as antisense oligonucleotides 17-35 nucleosides in length, such as 20-30 nucleosides in length, wherein the antisense oligonucleotide comprises a contiguous nucleotide sequence comprising at least 12, such as at least 13, such as at least 14, such as at least 15, such as at least 16, such as at least 17, such as at least 18 contiguous nucleotides complementary to the sequence present in SEQ ID NO 1.

The disclosure provides antisense oligonucleotides according to the disclosure, such as antisense oligonucleotides 17-35 nucleosides in length, such as 20-30 nucleosides in length, wherein the antisense oligonucleotide comprises a contiguous nucleotide sequence comprising at least 12, such as at least 13, such as at least 14, such as at least 15, such as at least 16, such as at least 17, such as at least 18 contiguous nucleotides present in SEQ ID NO 2.

According to another aspect of the disclosure there is provided an antisense oligonucleotide, 17-35 nucleobases long, capable of binding to the 5′ untranslated region (5′-UTR) of SMN2.

According to another aspect of the disclosure there is provided an antisense oligonucleotide, 17-35 nucleobases long, which is capable of specifically hybridizing to a target region sequence having a sequence disclosed in SEQ ID NO: 1, or a sequence with 90% identity thereto.

According to another aspect of the disclosure there is provided an antisense oligonucleotide, 17-35 nucleobases long, wherein said oligonucleotide comprises a base sequence that has at least 80% sequence identity with a sequence that is complementary to a target region sequence that is located at chr5:70049572-70049604 in UCSC hg38; or a pharmaceutically acceptable salt thereof.

According to another aspect of the disclosure there is provided an antisense oligonucleotide, 17-35 nucleobases long, wherein said oligonucleotide comprises a base sequence that has at least 80% sequence identity with a sequence disclosed in SEQ ID NO: 2; or a pharmaceutically acceptable salt thereof.

In other embodiments, the oligonucleotide comprises or consists of a base sequence that is complementary to the target region sequence disclosed in SEQ ID NO: 1, except for the presence of 1-5 nucleobase substitutions. Thus, for example an oligonucleotide that is 20 bases long may complement the target region sequence having a sequence as disclosed in SEQ ID NO: 1 at 15 or more base locations.

The oligonucleotide is advantageously an antisense oligonucleotide, which is typically single stranded or predominantly single stranded.

SEQ ID NO:1 provides the sequence of part of the 5′UTR of SMN2 and a few additional bases upstream. In the context of this application this region is referred to as the “target region” and this sequence is referred to as the “target region sequence”. SEQ ID NO: 2 provides the complement sequence to SEQ ID NO: 1.

The oligonucleotide of the disclosure is capable of hybridizing to all or part of the target region nucleic acid in a cell. Following such hybidization/binding, the cell is capable of increased expression of SMN2 mRNA and/or decreased mRNA degradation and/or increased total SMN protein expression. In one embodiment, the oligonucleotides of the present disclosure hybridize to the target region sequence with estimated ΔG° values below −10 kcal.

The disclosure further provides for an antisense oligonucleotide, 20-30 nucleotides in length, wherein said antisense oligonucleotide comprises a contiguous nucleotide sequence 20-30 nucleotides in length, wherein the contiguous nucleotide sequence is at least 90% complementary to SEQ ID No: 1. Advantageously, the antisense oligonudeotide is capable of effecting an enhanced level of expression of SMN2 mRNA and/or protein in a cell which is expressing SMN2 mRNA and/or protein. Advantageously, the antisense oligonucleotide is capable of effecting an enhanced level of expression of SMN1 mRNA and/or protein in a cell which is expressing SMN1 mRNA and/or protein, e.g. in cells without inactivating mutations in SMN1.

The oligonucleotides targeting SMN2 5′UTR may be antisense oligonucleotides that are capable of hybridizing to the SMN1/SMN2 target region nucleic acid.

The disclosure can be utilized in any mammalian species that expresses SMN protein. Advantageously, the disclosure is carried out in a human or using human cells.

The oligonucleotides of the disclosure can be capable of increasing the amount of human SMN2 mRNA produced in a cell. Indirectly, this leads to an increase in human SMN protein level. In patients that suffer from 5q spinal muscular dystrophy (SMA), i.e., SMA caused by mutations in the survival motor neuron 1 gene (SMN1), insufficient full-length functional survival motor neuron protein (SMN) is produced. By increasing the overall amount of total protein produced more full-length protein will be produced.

In some embodiments, the oligonucleotide of the disclosure is capable of increasing the expression of SMN2 in a cell which is expressing said SMN2.

In some embodiments, the oligonucleotide of the disclosure is capable of increasing the expression of SMN1 in a cell which is expressing said SMN1.

It is recognized that adenine bases in a target sequence can complement with thymine or uracil bases. Accordingly, throughout this specification it is understood that any thymine base in an oligonucleotide of the disclosure can be replaced by a uracil base.

In one aspect, the present disclosure is directed to an isolated oligonucleotide sequence comprising the sequence 5′-GTTATCGCCCTCCCACATTTGTGG-3′ (SEQ ID NO: 3; ASO #1), or as sequence with at least 80%, such as at least 90%, sequence identity thereto. In another aspect, the present disclosure is directed to an isolated oligonucleotide sequence comprising the sequence 5′-TGGTTATCGCCCTCCCACATTTGT-3′ (SEQ ID NO: 4; ASO #2) or as sequence with at least 80%, such as at least 90%, sequence identity thereto. In a further aspect, the present disclosure is directed to an isolated oligonucleotide sequence comprising the sequence 5′-AGTGGTTATCGCCCTCCCACATTT-3′ (SEQ ID NO: 5; ASO #3) or as sequence with at least 80%, such as at least 90%, sequence identity thereto. In a further aspect, the present disclosure is directed to an isolated oligonucleotide sequence comprising the sequence 5′-3′ (SEQ ID NO: 6; ASO #4) or as sequence with at least 80%, such as at least 90%, sequence identity thereto. In a further aspect, the present disclosure is directed to an isolated oligonucleotide sequence comprising the sequence 5′-3′ (SEQ ID NO: 7; ASO #5) or as sequence with at least, such as at least 90%, sequence identity thereto.

In one aspect, the disclosure provides for an oligonucleotide, 17-35 nucleotides in length, wherein said oligonucleotide comprises a contiguous nucleotide sequence of 10-33 nucleotides in length, wherein the contiguous nucleotide sequence is at least 90% complementary to SEQ ID No: 1; or a pharmaceutically acceptable salt thereof.

In particular embodiments, the oligonucleotide of the disclosure comprises one or more intemucleoside linkages between the contiguous nucleotides. Advantageously, the intemucleotide linkage is a phosphorothioate intemucleoside linkage.

In a particular embodiment, all the internucleoside linkages can be between the contiguous nucleotides in the oligonucleotide of the disclosure are phosphorothioate intemucleoside linkages.

In particular embodiments, the oligonucleotide of the disclosure is an antisense oligonucleotide comprising a nucleobase sequence selected from the group consisting of: SEQ ID Nos 3-7, wherein all inter-nucleoside linkages in the contiguous nucleoside sequence are phosphorothioate inter-nucleoside linkages, and optionally each DNA cytosine may be 5-methyl cytosine.

In particular embodiments, the oligonucleotide of the disclosure comprises one or more modifications, such as one or more modified sugar moieties or phosphate groups. Thus, in particular embodiments at least one sugar moiety and/or at least one phosphate bond moiety in the oligonucleotide is modified.

In particular embodiments, the oligonucleotide of the disclosure comprises one or more modified sugar moieties. The modified sugar moiety is typically a ribose in which (i) the —OH group at the 2′-position is substituted with any group selected from the group consisting of OR, R, R′OR, SH, SR, NH₂, NHR, NR₂, N₃, CN, F, Cl, Br and I (wherein R represents alkyl or aryl, and R′ represents alkylene) or 2′-F-ANA or (ii) the modified sugar moiety is a constrained ethyl (cET), an ethylene bridge nucleic acid (ENA) or a locked-nucleic acid (LNA) modification.

In particular embodiments, one or more sugar moieties in the oligonucleotide substituted at the 2′ position with any group selected from the group consisting of: 2′-O-(2-methoxyethyl) (2-MOE) or 2′-O-methyl (2′-OMe).

In particular embodiments, the oligonucleotide of the disclosure comprise one or more phosphate bond moiety selected from the group consisting of a phosphorothioate bond, a phosphorodithioate bond, an alkylphosphonate bond, a phosphoroamidate bond and a boranophosphate bond.

In particular embodiments, the oligonucleotide of the disclosure comprises at least one morpholino ring. Advantageously, the oligonucleotide of the disclosure is a morpholino oligomer or phosphorodiamidate morpholino oligomer.

In particular embodiments the oligonucleotide of the disclosure is modified with one or more 2′-OMe bases and one or more phosphorothioate linkages.

In particular embodiments the oligonucleotide of the disclosure is fully modified with 2′-OMe bases phosphorothioate linkages.

In particular embodiments the oligonucleotide of the disclosure is modified with one or more 2′-MOE bases and one or more phosphorothioate linkages.

In particular embodiments the oligonucleotide of the disclosure is fully modified with 2′-MOE bases and phosphorothioate linkages.

The oligonucleotide of the disclosure as referred to or claimed herein may be in the form of a pharmaceutically acceptable salt, such as a sodium salt or a potassium salt.

The oligonucleotide of the disclosure is one which when bound to the 5′UTR of SMN2 in a cell is capable of increasing the expression of SMN2 in the cell. In particular embodiments increased expression is of SMN2 mRNA and/or total protein and/or full-length protein.

### Method of Manufacture

The disclosure provides methods for manufacturing the oligonucleotides of the disclosure comprising reacting nucleotide units and thereby forming covalently linked contiguous nucleotide units comprised in the oligonucleotide. Preferably, the method uses phophoramidite chemistry (see, for example, Caruthers et al, 1987, Meth. Enzymol. vol. 154, pp 287-313). The method can further comprise reacting the contiguous nucleotide sequence with a conjugating moiety (ligand) to covalently attach the conjugate moiety to the oligonucleotide. In a further aspect a method is provided for manufacturing the composition of the disclosure, comprising mixing the oligonucleotide or conjugated oligonucleotide of the disclosure with a pharmaceutically acceptable diluent or carrier.

### Pharmaceutical Salts

The oligonucleotides according to the present disclosure may exist in the form of their pharmaceutically acceptable salts. The term “pharmaceutically acceptable salt” refers to conventional acid-addition salts or base-addition salts that retain the biological effectiveness and properties of the oligonucleotides molecules of the present disclosure and are formed from advantageous non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound (e.g. oigonucleotides molecule) into a salt is a technique well known to pharmaceutical chemists in order to obtain improved physical and chemical stability, hygroscopicity, flowability and solubility of compounds. It is for example described in Bastin, Organic Process Research & Development (2000) 4: 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt or a potassium salt.

### Pharmaceutical Composition

The disclosure furthers provides embodiments of a pharmaceutical composition comprising the oligonucleotide of the disclosure and a pharmaceutically acceptable diluent or carrier. The pharmaceutical composition may be in lyophilized form.

A pharmaceutically acceptable diluent includes phosphate-buffered saline (PBS). In some embodiments the pharmaceutically acceptable diluent is sterile phosphate buffered saline.

The pharmaceutical composition of the present disclosure may optionally comprise a pharmaceutically acceptable additive, in addition to the antisense oligonucleotide of the present disclosure or a pharmaceutically acceptable salt thereof and/or a carrier as described above. Examples of such an additive include an emulsifier aid (e.g., a fatty acid containing 6 to 22 carbon atoms or a pharmaceutically acceptable salt thereof, albumin, dextran), a stabilizing agent (e.g., cholesterol, phosphatidic acid), an isotonizing agent (e.g., sodium chloride, glucose, maltose, lactose, sucrose, trehalose), and a pH adjuster (e.g., hydrochloric acid, sulfuric acid, phosphoric acid, acetic acid, sodium hydroxide, potassium hydroxide, triethanolamine). These additives may be used either alone or in combination. The content of the additive(s) in the pharmaceutical composition of the present disclosure is reasonably 90% by weight or less, such as 60% by weight or less, and advantageously 50% by weight or less.

The pharmaceutical composition of the present disclosure may be prepared by adding the compound (e.g. antisense oligonucleotide) of the present disclosure or a pharmaceutically acceptable salt thereof to a dispersion of a carrier, followed by adequate stirring. The additive(s) may be added at any appropriate stage, either before or after adding the compound of the present disclosure or a pharmaceutically acceptable salt or hydrate thereof. Any aqueous solvent may be used for adding the compound of the present disclosure or a pharmaceutically acceptable salt or hydrate thereof as long as it is pharmaceutically acceptable, and examples include injectable water, injectable distilled water, electrolytic solutions (e.g., physiological saline), and sugar solutions (e.g., glucose solution, maltose solution). Moreover, in this case, conditions including pH and temperature may be selected as appropriate by those skilled in the art.

Advantageous formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further advantageous and preferred examples of pharmaceutically acceptable diluents and carriers (hereby incorporated by reference). Advantageous dosages, formulations, administration routes, compositions, dosage forms, combinations with other therapeutic agents, pro-drug formulations are also provided in WO02007/031091.

The pharmaceutical composition of the present disclosure may be formulated into a solution or a lyophilized formulation thereof. Such a lyophilized formulation may be prepared in a standard manner by freeze-drying the pharmaceutical composition of the present disclosure in a solution form. For example, the pharmaceutical composition of the present disclosure in a solution form may be sterilized as appropriate (e.g. by conventional sterilization or sterile filtration techniques) and then dispensed in given amounts into vial bottles, followed by preliminary freezing under conditions of about −40° C. to −20° C. for about 2 hours, primary drying at about 0° C. to 10° C. under reduced pressure and then secondary drying at about 15° C. to 25° C. under reduced pressure. Moreover, in most cases, the vials may be purged with a nitrogen gas and then capped, thereby giving a lyophilized formulation of the pharmaceutical composition of the present disclosure.

Such a lyophilized formulation of the pharmaceutical composition of the present disclosure may generally be used after being reconstituted by addition of any appropriate solution (i.e., a reconstituting solution). Examples of such a reconstituting solution include injectable water, physiological saline, and other commonly used infusion solutions. The volume of such a reconstituting solution will vary, e.g., depending on the intended use and is not limited in any way, but it is reasonably 0.5- to 2-fold greater than the solution volume before freeze-drying, or 500 mL or less.

The pharmaceutical composition of the present disclosure may be administered in any pharmaceutically acceptable mode, which may be selected as appropriate for the intended therapeutic method. Advantageous methods include intravenous administration, intraarterial administration, intramuscular administration, subcutaneous administration, oral administration, interstitial administration, percutaneous administration and so on. Moreover, the composition of the present disclosure may be in any dosage form, and examples include various types of injections, oral formulations, drops, inhalants, ointments, lotions, etc.

### Applications

The oligonucleotides of the disclosure may be utilized as research reagents for, for example, diagnostics, therapeutics and prophylaxis.

In research, such oligonucleotides may be used to specifically modulate the synthesis of SMN protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein. If employing the oligonucleotide of the disclosure in research or diagnostics the target nucleic acid may be a cDNA or a synthetic nucleic acid derived from DNA or RNA.

The present disclosure provides an in vivo or in vitro method for modulating SMN2 expression in a target cell which is expressing SMN2, said method comprising administering an oligonucleotide of the disclosure in an effective amount to said cell.

In some embodiments, the target cell, is a mammalian cell such as a human, a monkey or murine cell. The target cell may be an in vitro cell culture or an in vivo cell forming part of a tissue in a mammal.

In diagnostics the oligonucleotides may be used to detect and quantitate SMN2 expression in cell and tissues by northern blotting, in-situ hybridisation or similar techniques.

For therapeutics, the oligonucleotides of the disclosure can be used to modulate the expression of SMN2 in an animal (e.g. a human) suspected of having a disease or disorder mediated by or associated with aberrant SMN1 expression.

In a particular embodiment, an oligonucleotide of the disclosure is used to increase the expression of SMN2 in an animal suspected of having a disease or disorder mediated by or associated with aberrant SMN2 expression.x In an embodiment, the disease or disorder is one mediated by or associated with reduced amounts of full length SMN protein in the affected cells. In some embodiments the disease or disorder may be associated with a mutation in the SMN1 gene. SMA and SLA are diseases mediated by aberrant levels of SMN protein in the cell.

As demonstrated in the Examples, treatment with agents of the disclosure increases the amount of total SMN2 mRNA present in a cell. Since it is known that a certain proportion of SMN2 mRNA (between around 10% and 20%, depending on the individual) can give rise to full-length functional SMN protein, the ability to increase the total amount of SMN2 mRNA present in a cell will enable an increased overall cellular production of SMN protein. Thus, the agents of the disclosure, and methods using these agents, are able to offer a therapeutic approach for the prevention and/or treatment of diseases or conditions mediated by, or associated with, reduced SMN protein levels.

Furthermore, a number of therapies have been developed that facilitate splice-switching during the processing of SMN2 mRNA to enable production of full-length SMN protein from a far higher proportion of the total number of SMN2 mRNA transcripts produced. Treatment of cells with the agents of the disclosure to increase the total quantity of SMN2 mRNA present thus provides a larger “pool” of SMN2 transcripts upon which these splice-switching agents can work. Alternatively, the 5′UTR ASO (oligonucleotide of the disclosure) could act secondarily to the splice switching agent, enhancing the stability or translation of already spliced mRNA in the cytoplasm. In either series of events, the 5′UTR ASO is overcoming the ceiling effect of the splice-switching agent and further increasing the production of SMN protein. The Examples demonstrate that treatment of cells with both agents of the disclosure and a substance that promotes splice-switching enables the production of larger quantities of SMN protein than does treatment with either of these agents individually. Accordingly, it will be recognized that combination therapy using agents of the disclosure in conjunction with substances that promote splice-switching in the processing of SMN2 mRNA offers a new possibility for therapeutic intervention that is both new and surprisingly effective.

The disclosure provides for an in vivo or in vitro method for modulating SMN2 expression in a target cell which is expressing SMN2, said method comprising administering an oligonucleotide or pharmaceutical composition of the disclosure in an effective amount to said cell.

The disclosure provides a method of promoting the transcription of SMN2 in a subject in need thereof, the method comprising providing to the subject an effective amount of an agent of the disclosure.

The disclosure provides a method of inhibiting degradation of SMN2 mRNA in a subject in need thereof, the method comprising providing to the subject an effective amount of an agent of the disclosure.

The disclosure provides a method of therapeutically increasing steady-state levels of SMN2 mRNA in a subject in need thereof, the method comprising providing to the subject an effective amount of an agent of the disclosure.

In view of the explanation above, it will be recognized that the methods of the disclosure are all capable of therapeutic utility, particularly in the context of combination therapies (such as with substances that promote splice-switching) by virtue of their respective abilities to promote transcription of SMN2, inhibit degradation of SMN2 mRNA, and increase steady-state levels of SMN2 mRNA.

The disclosure provides for a method for treating or preventing a disease comprising administering a therapeutically or prophylactically effective amount of an oligonucleotide or a pharmaceutical composition of the disclosure to a subject suffering from or susceptible to the disease. In some embodiments, the disease is one that is mediated by or associated with decreased SMN protein production.

In some embodiments, the disease is selected from the group consisting of: SMA and ALS.

In some embodiments, the disease or condition is one that will benefit from increased SMN2 mRNA production or full-length SMN protein production.

The oligonucleotides of the disclosure are capable of effecting an increase in the amount of SMN2 mRNA and/or protein in a cell which is expressing the SMN2 target nucleic acid, such as the SMN2 mRNA.

In some embodiments, oligonucleotides of the disclosure are capable of increasing the expression of SMN2 target nucleic acid in a cell which is expressing the target nucleic acid, so to increase the amount of SMN2 target nucleic acid (e.g. the mRNA) by at least 50%, at least 60%, at least 70%, at least 80%, or at least 90% compared to the expression level of the SMN2 target nucleic acid (e.g. the mRNA) in the cell in the absence of the agent.

The disclosure provides methods for treating or preventing a disease, comprising administering a therapeutically or prophylactically effective amount of an oligonucleotide or a pharmaceutical composition of the disclosure to a subject suffering from or susceptible to the disease.

The disclosure also relates to an oligonucleotide or a pharmaceutical composition as defined herein for use as a medicament.

The oligonucleotide or a pharmaceutical composition according to the disclosure is typically administered in an effective amount.

In a particular embodiment, the patient is identified as having a disease or condition characterized by aberrant (e.g. lower than normal) SMN protein levels prior to administration of the oligonucleotide or pharmaceutical composition according to the disclosure. Such identification can be carried out according to a variety of methods as described herein. In certain embodiments, the amount or level of full-length SMN protein is determined from a biological sample previously isolated from the patient/subject. Such sample could be a biopsy (such as tumor tissue) or fluid (such as blood) sample.

There are various well-known methods for determining the amount of protein or mRNA in a cell. Immunohistochemistry, ELISA, or mass spectroscopy methods, such as liquid-chromatography mass spectroscopy (LC-MS) are particularly advantageous methods.

For mRNA determination, methods involving hybridization to the target mRNA using a complementary nucleic acid can be employed. Various adaptations of reverse transcription polymerase chain reaction (RT-PCR), such as quantitative PCR or competitive RT-PCR, are advantageous quantitative methods for determining the relative amount of a mRNA species in a normal cell versus an aberrant cell. The person skilled in the art is able to employ an advantageous method for detection of the amount or protein or mRNA in the cell or cells.

The disclosure further relates to use of an oligonucleotide or a pharmaceutical composition as defined herein for the manufacture of a medicament for the treatment of abnormal levels and/or activity of SMN2. As noted above, such abnormal levels may be reduced levels.

In one embodiment, the disclosure relates to an oligonucleotide or a pharmaceutical composition comprising one of these in accordance with the disclosure for use in the treatment of diseases or disorders selected from: SMA and ALS.

### Administration

The oligonucleotides or pharmaceutical compositions of the present disclosure may be administered topical or enteral or parenteral (such as, intravenous, subcutaneous, intramuscular, intracerebral, intracerebroventricular or intrathecal).

The oligonucleotide or pharmaceutical compositions of the present disclosure are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In an advantageous embodiment of the methods of treatment of the disclosure, the agent of the disclosure, optionally in the form of a pharmaceutical composition of the disclosure, is provided directly to the central nervous system of the subject.

The agent can be provided to the cerebrospinal fluid of the subject. In an advantageous embodiment the agent may be provided to the subject by injection. For example, the agent may be provided to the subject by intrathecal injection.

In the case of a combination treatment, the further therapeutic agent, such as a substance that promotes splice-switching, may also be provided by injection. Such routes of administration are advantageous for use in embodiments in which the substance that promotes splice-switching comprises an oligonucleotide, such as nusinersen.

Additionally, or alternatively, the substance that promotes splice-switching may be provided to the subject by oral administration. Such embodiments are particularly advantageous when the is substance that promotes splice-switching is selected from the group consisting of: risdiplam and branaplam.

### Dosages

The agents and pharmaceutical compositions of the disclosure are for provision in a an “effective” or “therapeutically effective” amount. Except for where the context requires otherwise, references to effective amounts and therapeutically effective amounts should be considered as interchangeable for the purposes of the present disclosure.

An effective or therapeutically effective amount is the amount of an agent or composition that alone, or together with further doses, produces a desired response.

In some embodiments an agent of the disclosure, such as an oligonucleotide, is used in the pharmaceutically acceptable diluent at a concentration of 50-300 μM solution.

In some embodiment, an agent of the disclosure, such as an oligonucleotide, is provided in a therapeutically effective amount of between about 5 mg and about 50 mg. Advantageously the agent is provided in a therapeutically effective amount of between about 10 mg and about 15 mg.

In some embodiments, an agent of the disclosure, such as an oligonucleotide, is administered at a dose of 10-1000 μg.

In some embodiments, an agent of the disclosure, such as an oligonucleotide, oligonucleotide conjugate or pharmaceutical composition of the disclosure is administered at a dose of 0.1-15 mg/kg, such as from 0.2-10 mg/kg, such as from 0.25-5 mg/kg. The administration can be once a week, every 2^{nd} week, every third week, once a month or every 4 or 6 months.

### Transcription of SMN2

The disclosure further provides methods capable of bringing about a desired promotion of transcription of SMN2 in a subject. By transcription is meant the production of mRNA or pre-mRNA.

Promotion of transcription of SMN2 in a subject may be demonstrated by an increase in the quantity of SMN2 mRNA or pre-mRNA present. Quantities of SMN2 mRNA or pre-mRNA may be assessed by any advantageous method, including the use of qRT-PCR using primers for SMN, or RNA sequencing. In the context of the methods of the disclosure, an effective amount of an agent of the disclosure may be an amount able to promote transcription of SMN2 by at least 5%, at least 10%, at least 15%, or at least 20%. Advantageously, an effective amount of an agent of the disclosure may be an amount able to promote transcription of SMN2 by at least 25%, at least 30%, at least 35%, at least 45%, at least 50%, at least 55%, at least 65%, at least 70%, or at least 75%. Indeed, an effective amount of an agent of the disclosure may be an amount able to promote transcription of SMN2 by at least 80%, at least 85%, at least 90%, at least 95%, or by 100% or more.

Inhibition of Degradation of SMN2 mRNA

The disclosure further provides methods capable of bringing about a desired inhibition of degradation of SMN2 mRNA in a subject.

Degradation of SMN2 mRNA may be assessed with reference by methods such as actinomycin D studies, in which cells treated with an agent of the disclosure (for example with a putative dose of an agent of the disclosure) are then treated with actinomycin D, and cellular levels of SMN2 mRNA assessed over time. Details of such studies are set out in the Examples below. Alternative methods, including pulse-chase experiments using nucleoside analogues will also be known to those skilled in the art.

In the context of the methods of the sixth aspect of the disclosure, an effective amount of an agent of the disclosure may be an amount able to inhibit degradation of SMN2 mRNA by at least 5%, at least 10%, at least 15%, or at least 20%. Advantageously, an effective amount of an agent of the disclosure may be an amount able to inhibit degradation of SMN2 mRNA by at least 25%, at least 30%, at least 35%, at least 45%, at least 50%, at least 55%, at least 65%, at least 70%, or at least 75%. Indeed, an effective amount of an agent of the disclosure may be an amount able to inhibit degradation of SMN2 mRNA by at least 80%, at least 85%, at least 90%, at least 95%, or by 100% or more. Inhibition of degradation of SMN2 mRNA may be determined with reference to advantageous controls.

Steady-State Levels of SMN2 mRNA

Embodiments of the disclosure are further directed to methods to increase in steady-state levels of SMN2 mRNA in a subject cell or population of cells. It is generally understood that steady-state levels refer to total levels of the transcript of interest in a cell at any given time. This is a product of the rate of transcription and the rate of degradation. Steady-state levels of SMN2 mRNA may be assessed by qRT-PCR using primers for SMN, or RNA sequencing.

In the context of the methods of the disclosure to increase in steady-state levels of SMN2 mRNA in a subject cell or population of cells, an effective amount of an agent of the disclosure can be an amount able to increase steady-state SMN2 mRNA levels by at least 5%, at least 10%, at least 15%, or at least 20%. Advantageously, an effective amount of an agent of the disclosure may be an amount able to increase steady-state SMN2 mRNA levels by at least 25%, at least 30%, at least 35%, at least 45%, at least 50%, at least 55%, at least 65%, at least 70%, or at least 75%. An effective amount of an agent of the disclosure can be an amount able to increase steady-state SMN2 mRNA levels by at least 80%, at least 85%, at least 90%, at least 95%, or by 100% or more.

### Prevention or Treatment of SMA

Further provided are methods for the treatment of SMA in a subject. These methods utilize therapeutically effective amounts of agents of the disclosure. Effectiveness of treatment of SMA may be demonstrated by improvement in at least one clinical assessment of SMA, or the reduction of at least one pathological cause of SMA. Merely by way of example, any of the following measurements may be used to assess effectiveness of an SMA therapy: Hammersmith Functional Motor Scale (HFMS), Revised Upper Limb Module (RULM), Children's Hospital of Philadelphia Infant Test of Neuromuscular Disorders (CHOP INTEND), 6 Minute Walk Test (6MWT), or Pulmonary Function Tests.

A therapeutically effective amount of an agent of the disclosure can be an amount able to bring about a stabilization of disease or lack of disease progression or a clinical improvement in SMA by at least 5%, at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 95%, or by 100% or more. Effectiveness of treatment of SMA may be determined with reference to suitable comparators, including clinically available guides for the assessment of the pathological causes or symptoms of SMA.

### Combination Therapies

In some embodiments of the disclosure the oligonucleotide or pharmaceutical composition of the disclosure is for use in a combination treatment with at least one other therapeutic agent. The therapeutic agent can, for example, be the standard of care for the diseases or disorders described above.

Representative agents (and company developing or commercializing the agent) approved or in clinical development for the treatment of SMA and/or ALS, while not intended to be limiting, may be considered for use in combination with the agent9s) of the disclosure: leuproline acetate SR (Takeda Pharmaceuticals Co. Ltd.), nusinersen (Biogen), adenosine (Kowa Co.), onasemnogene abeparvovec (Avexis Inc.), amifampridine phosphate (Catalyst Pharmaceuticals), branaplam (Novartis), valproate sodium (Kowa Co.), risdiplam (Roche), reidesemtiv (Cytokinetics Inc.), BIIB-110 (Biogen Inc.), ARM-210 (Armgo Pharma), AAD-2004 (GNT Pharma) and SRK-105 (Scholar Rock).

Representative agents (and company developing or commercializing the agent) undergoing pre-clinical development for the treatment of SMA and/or ALS, while not intended to be limiting, may be considered for use in combination with the agent of the disclosure: maresin-1 (Ono Pharmaceuticals), ND-602 (Neurodyn Life Sciences), resagen (University of Sheffield), Xcel-hNuP (Xcelthera), SMN-2 (Exicure), ALG-802 (Biogen), NT-1654 (Neurotune AG), ALB-111 (National Institute of Neurological Disorders and Stroke), LDN-5178 (Spotlight Innovation), gene therapy (WO2008150509; Biogen), Xcel-hNu (Xcelthera), STL-182 (Spotlight Innovation), REC-001202 (Recursion Pharmaceuticals), REC-0000716 (Recursion Pharmaceuticals), PMO-25 (Sarepta), INT-41 (Vybion), and TEC-1 (Reboma Biosciences).

The therapeutic compositions and methods of the disclosure may be used in combination therapies. Further details of advantageous embodiments of such combination therapies are considered elsewhere in the present disclosure and include those in which an agent of the disclosure is provided to the subject in a combination therapy with a substance that promotes splice-switching.

Unless the context requires otherwise, when used in combination therapies the agent or agents of the disclosure may be provided to the subject by combined, sequential or separate administration with the further therapeutic agent, such as a substance that promotes splice-switching. In some advantageous embodiments, the substance that promotes splice-switching promotes exon 7 inclusion in SMN2 mRNA.

Merely by way of example, such a substance that promotes splice-switching may be, but is not limited to, nusinersen, risdiplam, or branaplam. Each of these is advantageous for use in combination treatments with the agents of the disclosure. The substance that promotes splice-switching is advantageously an oligonucleotide. For example, an oligonucleotide may comprise the nucleotide sequence TCACTTTCATAATGCTGG (SEQ ID No: 14). This sequence is found in nusinersen.

Other therapeutic agents that may be used in combination therapies with the agents of the disclosure can be, but are not limited to, those selected from the group consisting of: leuprolide acetate; adenosine; onasemnogene abeparvovec; amifampridine phosphate; valproate sodium; and reldesemtiv. Of these, leuprolide acetate, adenosine, and onasemnogene abeparvovec can all be administered by injection (most advantageously intramuscular, intravenous and intrathecal injection, respectively). Amifampridine phosphate, valproate sodium, and reldesemtiv can all be provided orally.

Therapeutic methods of treatment of the disclosure may be used in combination therapies with other known therapeutic agents or regimens used in the treatment of SMA. Merely by way of example, methods in accordance with the disclosure may be used in combination with one or more further therapies such as, but not limited to, gene therapy, potassium ion (K⁺) channel blockers, and GABA inhibitors.

Advantageous examples of gene therapy that may be used in combination with methods of treatment of the disclosure may be determined by one skilled in the art, and can include, for example, treatment with AVXS-101.

One aspect of the disclosure encompasses embodiments of an oligonucleotide having between about 17 to about 35 nucleotides in length, wherein said oligonucleotide can comprise a base sequence complementary to at least 10 consecutive bases of the nucleotide sequence SEQ ID NO: 1, or a pharmaceutically acceptable salt thereof.

In some embodiments of this aspect of the disclosure, the oligonucleotide can be an antisense oligonucleotide.

In some embodiments of this aspect of the disclosure, the oligonucleotide can be 20 to 30 nucleotides in length.

In some embodiments of this aspect of the disclosure, the base sequence can be complementary to at least 12-20 consecutive bases of the nucleotide sequence SEQ ID NO: 1.

In some embodiments of this aspect of the disclosure, the base sequence can be complementary to all of the nucleotide sequence SEQ ID NO: 1.

In some embodiments of this aspect of the disclosure, the at least one sugar moiety, at least one phosphate bond moiety, or at least one sugar moiety and at least one phosphate bond moiety of the oligonucleotide can be modified.

In some embodiments of this aspect of the disclosure, (i) the modified sugar moiety can be a ribose wherein the —OH group at the 2′-position is substituted with a group selected from the group consisting of OR, R, R′OR, SH, SR, NH₂, NHR, NR₂, N₃, CN, F, Cl, Br and I (wherein R is an alkyl or an aryl group, and R′ represents an alkylene group or 2′-F-ANA or (ii) the modified sugar moiety can be a constrained ethyl (cET), an ethylene bridge nucleic acid (ENA), or a locked-nucleic acid (LNA) modification.

In some embodiments of this aspect of the disclosure, the modified sugar moiety can be a ribose wherein —OH group at the 2′-position is substituted with 2′-O-(2-methoxyethyl) (2-MOE) or 2′-O-methyl (2′-OMe).

In some embodiments of this aspect of the disclosure, the modified phosphate bond moiety can be selected from the group consisting of a phosphorothioate bond, a phosphorodithioate bond, an alkylphosphonate bond, a phosphoroamidate bond, and a boranophosphate bond.

In some embodiments of this aspect of the disclosure, the antisense oligomer can comprise at least one morpholino ring.

In some embodiments of this aspect of the disclosure, the oligonucleotide can be a morpholino oligomer or a phosphorodiamidate morpholino oligomer.

In some embodiments of this aspect of the disclosure, the intemucleoside linkages of the contiguous nucleotide sequence can be phosphorothioate internucleoside linkages.

In some embodiments of this aspect of the disclosure, the oligonucleotide can comprise a sequence selected from the group consisting of: SEQ ID NO: 3, SEQ ID NO: 4, SEQ ID NO: 5, SEQ ID NO: 6, and SEQ ID NO: 7.

In some embodiments of this aspect of the disclosure, the oligonucleotide can be admixed with a pharmaceutically acceptable diluent or carrier.

Another aspect of the disclosure encompasses embodiments of a method for modulating the expression of SMN2 in a cell expressing SMN2, the method comprising the step of administering an oligonucleotide of claim 1 to the cell or a population of cells.

In some embodiments of this aspect of the disclosure, the population of cells can be in a patient having a pathological condition associated with a reduction in motor neuron or neuromuscular junction numbers or with a disruption of motor neurons or neuromuscular junctions, and wherein the modulation of the expression of SMN2 can be a treatment for, or a prevention of, the pathological condition of the recipient patient.

Yet another aspect of the disclosure encompasses embodiments of a method of increasing a steady-state level of an mRNA encoding SMN2 in a subject in need thereof, the method comprising providing to the subject a therapeutically effective amount of an oligonucleotide of claim 1, wherein the oligonucleotide can promote the transcription of mRNA encoding SMN2, or inhibit degradation of an mRNA encoding SMN2 in cells of the subject in need thereof.

In some embodiments of this aspect of the disclosure, the subject can have a condition associated with a reduction in motor neuron or neuromuscular junction numbers, or a condition associated with disruption of motor neurons or neuromuscular junctions.

In some embodiments of this aspect of the disclosure, the subject ca have spinal muscular atrophy, has 5q spinal muscular atrophy, can be a geriatric subject, can have an acute muscle injury, or can have a chronic muscle injury.

In some embodiments of this aspect of the disclosure, the oligonucleotide can be co-administered with a splice-switching promoter, and wherein the oligonucleotide and the splice-switching promoter can be administered to the patient simultaneously or sequentially.

In some embodiments of this aspect of the disclosure, the splice-switching promoter can promote exon 7 inclusion in SMN2 mRNA.

In some embodiments of this aspect of the disclosure, the splice-switching promoter can be selected from the group consisting of nusinersen, risdiplam, branaplam, or an oligonucleotide.

In some embodiments of this aspect of the disclosure, the oligonucleotide can comprise the nucleotide sequence of SEQ ID No: 14.

In some embodiments of this aspect of the disclosure, the splice-switching promoter can be nusinersen.

In some embodiments of this aspect of the disclosure, the oligonucleotide can be provided directly to the central nervous system of the subject.

In some embodiments of this aspect of the disclosure, the oligonucleotide can be provided directly to the cerebrospinal fluid of the subject.

In some embodiments of this aspect of the disclosure, the oligonucleotide can be provided to the subject by injection or by intrathecal injection.

In some embodiments of this aspect of the disclosure, the splice-switching promoter can be provided by injection.

In some embodiments of this aspect of the disclosure, the splice-switching promoter can be selected from risdiplam and branaplam, and is provided by oral administration.

In some embodiments of this aspect of the disclosure, the oligonucleotide can be provided in an amount of between about 5 mg to about 50 mg or between about 10 mg and about 15 mg.

Now having described the embodiments of the present disclosure, in general, the following examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### EXAMPLES

### Example 1

### Experimental Details and Results:

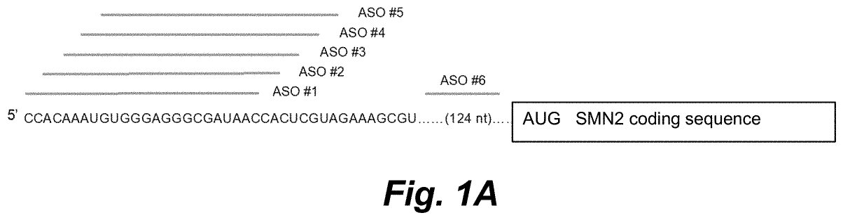

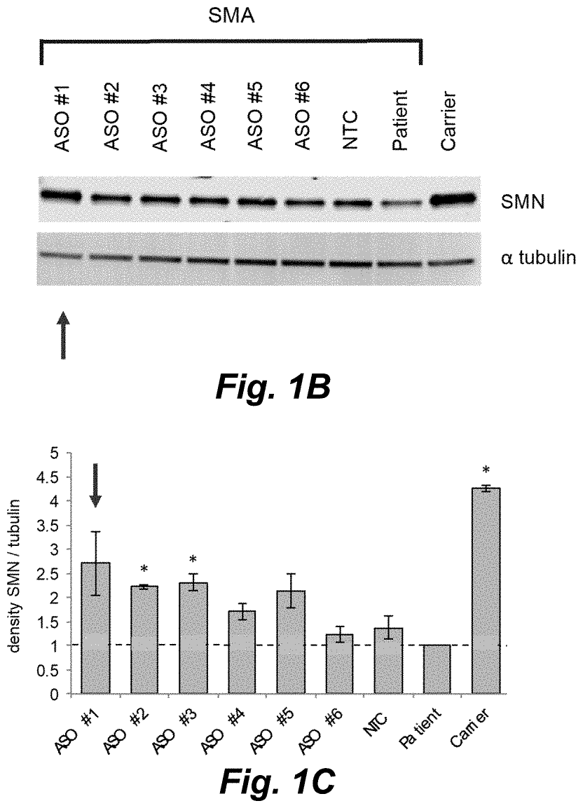

Oligonucleotides targeting the 5′UTR of SMN2 were designed. FIG. 1A shows the sequence and binding position of each antisense oligonucleotide (AON).

### Antisense Oligonucleotide(ASO) Synthesis:

All antisense oligonucleotides were purchased from Integrated DNA Technologies (IDT). The two types of ASOs used were:
(1) fully modified with 2′-O-Methyl (2′-OMe) bases and phosphorothioate linkages; or
(2) fully modified with 2′-O-(2-methoxyethyl) (2′-MOE) bases and phosphorothioate linkages.

Non-targeting control oligonucleotide (NTC):
(SEQ ID NO: 13)
5′-CCTCTTACCTCAGTTACAATTTATA
Splice-switching oligonucleotide:
(SEQ ID NO: 14)
5'-TCACTTTCATAATGCTGG-3′

corresponds to “ASO 10-27” (Hua et al., Am. J. Hum. Genet. 82: 834-848, 2008). Cell culture: SMA patient and carrier fibroblasts (Coriell GM00232 and GM03814, respectively) were cultured in DMEM supplemented with 10% FBS and maintained in a 37° C. incubator with 5% CO₂.

Antisense oligonucleotide transfections were performed in 6-well plates using RNAiMAX transfection reagent (Invitrogen). Media was changed one day post-transfection, and cells were harvested two days post-transfection, unless otherwise specified.

For mRNA stability assays, fibroblasts were transfected as described above, and then treated with 5 μg/mL actinomycin D (Sigma Aldrich) two days after transfection. Cells were then collected at the indicated time points post treatment with actinomycin D.

### Immunoblotting:

Lysates were prepared in RIPA buffer (50 mM Tris-HCl pH 8.0, 150 mM NaCl, 5 mM EDTA, 1% NP-40 (IGEPAL), 1% sodium deoxycholate, 0.1% SDS). 15 μg protein per sample were resolved on Novex 4-20% Tris-Glycine WedgeWell gels (Invitrogen) and transferred to PVDF membrane. Membranes were blocked with 5% milk containing Tris-buffered saline and 0.1% Tween before incubation with primary antibodies: mouse anti-SMN (BD Biosciences 610647), rabbit anti-alpha tubulin (Abcam ab4074; or Cell Signaling 2144). These were followed by incubations with either IRDye or HRP secondary antibodies and detected on a LI-COR Odyssey or using chemiluminescence detection (ChemiDoc), respectively. Densitometric analysis of protein signal was done using ImageJ software. mRNA expression analysis: Fibroblasts were collected in 500 μL TRIzol (Invitrogen). After adding 100 μL chloroform, samples were vortexed and centrifuged (15 min, 12,000×g, 4° C.). 1.5 volumes of 100% ethanol were added to the supernatant. Samples were then pipetted into columns and RNA purification continued according to the miRNeasy Mini Kit manual (QIAGEN). 1 μg total RNA was converted into cDNA using the High Capacity cDNA Reverse Transcription Kit (Applied Biosystems). 10 μL qRT-PCR reactions were performed using Power Sybr Green (Applied Biosystems), 4.2 μL cDNA (diluted 1:25) and 200 nM of each target primer:

Total SMN qF:
(SEQ ID NO: 15)
5′-GCGATGATTCTGACATTTGG;
Total SMN qR:
(SEQ ID NO: 16)
5′-GGAAGCTGCAGTATTCTTCT;
GAPDH qF:
(SEG ID NO: 17)
5′-CTCAACGACCACTTTGTCAAGCTC;
GAPDH qR:
(SEQ ID NO: 18)
5′-TCTTACTCCTTGGAGGCCATGT.

The annealing temperature for all qRT-PCR reactions was 60° C.

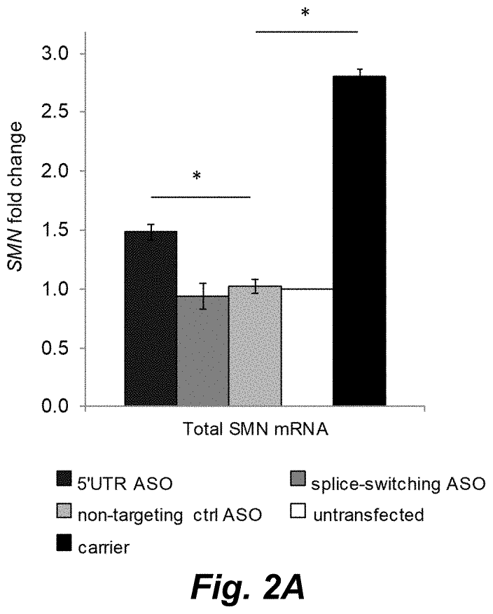

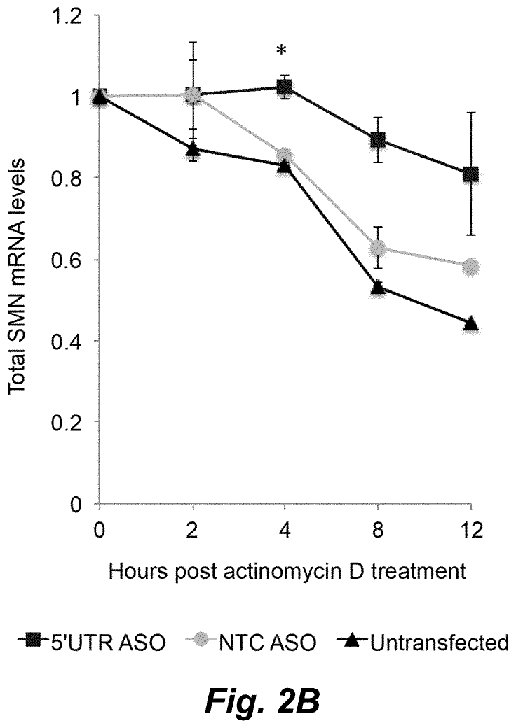

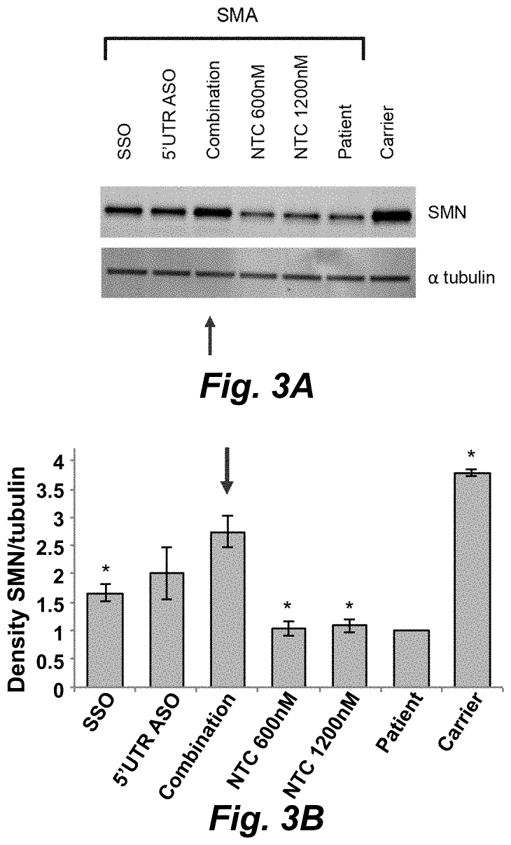

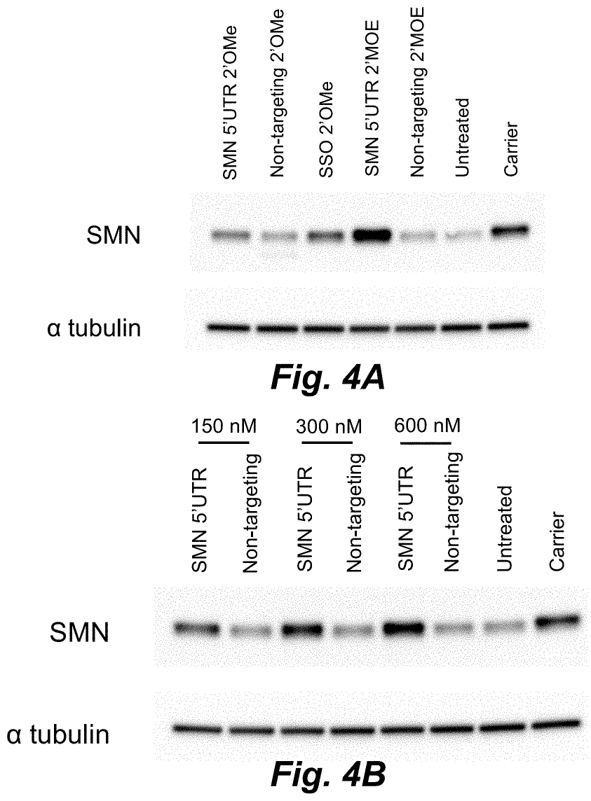

The results are shown in FIGS. 1A-4B.

### Example 2

| SEQ ID | | |
|---|---|---|
| NO. | Identity | Sequence |
| 1 | SMN2 target region | CCACAAATGTGGGAGGGCGATAACCACTCGTAG |
| 2 | SMN2 target region complement | CTACGAGTGGTTATCGCCCTCCCACATTTGTGG |
| 3 | ^{a}AON#1 | GTTATCGCCCTCCCACATTTGTGG |
| 4 | AON#2 | TGGTTATCGCCCTCCCACATTTGT |
| 5 | AON#3 | AGTGGTTATCGCCCTCCCACATTT |
| 6 | AON#4 | CGAGTGGTTATCGCCCTCCCACAT |
| 7 | AON#5 | TACGAGTGGTTATCGCCCTCCCAC |
| 8 | complement of SEQ ID NO: 3 | CCACAAATGTGGGAGGGCGATAAC |
| 9 | complement of SEQ ID NO: 4 | ACAAATGTGGGAGGGCGATAACCA |
| 10 | complement of SEQ ID NO: 5 | AAATGTGGGAGGGCGATAACCACT |
| 11 | complement of SEQ ID NO: 6 | ATGTGGGAGGGCGATAACCACTCG |
| 12 | complement of SEQ ID NO: 7 | GTGGGAGGGCGATAACCACTCGTA |
| 13 | Non-targeting Control (NTC) | CCTCTTACCTCAGTTACAATTTATA |
| 14 | Splice-switching oligonucleotide | TCACTTTCATAATGCTGG |
| 15 | Total SMN qForward Primer | GCGATGATTCTGACATTTGG |
| 16 | Total SMN qReverse Primer | GGAAGCTGCAGTATTCTTCT |
| 17 | GAPDH qForward Primer | CTCAACGACCACTTTGTCAAGCTC |
| 18 | GAPDH qReverse Primer | TCTTACTCCTTGGAGGCCATGT |

^{a}antisense oligonucleotide (AON)

1. An oligonucleotide having between about 17 to about 35 nucleotides in length, wherein said oligonucleotide comprises a base sequence complementary to at least 10 consecutive bases of the nucleotide sequence SEQ ID NO: 1, or a pharmaceutically acceptable salt thereof.

2. The oligonucleotide of claim 1, wherein the oligonucleotide is an antisense oligonucleotide.

3. The oligonucleotide according to claim 1, wherein the oligonucleotide is 20 to 30 nucleotides in length.

4. The oligonucleotide according to claim 1, wherein the base sequence is complementary to at least 12-20 consecutive bases of the nucleotide sequence SEQ ID NO: 1.

5. The oligonucleotide according to claim 1, wherein the base sequence is complementary to all of the nucleotide sequence SEQ ID NO: 1.

6. The oligonucleotide according to claim 1, wherein at least one sugar moiety, at least one phosphate bond moiety, or at least one sugar moiety and at least one phosphate bond moiety of the oligonucleotide is modified.

7. The oligonucleotide according to claim 6, wherein (i) the modified sugar moiety is a ribose wherein the —OH group at the 2′-position is substituted with a group selected from the group consisting of OR, R, R′OR, SH, SR, NH₂, NHR, NR₂, N₃, CN, F, Cl, Br and I (wherein R is an alkyl or an aryl group, and R′ represents an alkylene group or 2′-F-ANA or (ii) the modified sugar moiety is a constrained ethyl (cET), an ethylene bridge nucleic acid (ENA), or a locked-nucleic acid (LNA) modification.

8. The oligonucleotide according to claim 7, wherein the modified sugar moiety is a ribose wherein —OH group at the 2′-position is substituted with 2′-O-(2-methoxyethyl) (2-MOE) or 2′-O-methyl (2′-OMe).

9. The oligonucleotide according to claim 6, wherein the modified phosphate bond moiety is selected from the group consisting of a phosphorothioate bond, a phosphorodithioate bond, an alkylphosphonate bond, a phosphoroamidate bond, and a boranophosphate bond.

10. The oligonucleotide according to claim 1, wherein the antisense oligomer comprises at least one morpholino ring.

11. The oligonucleotide according to claim 10, wherein the oligonucleotide is a morpholino oligomer or a phosphorodiamidate morpholino oligomer.

12. The oligonucleotide according to claim 1, wherein the intemucleoside linkages of the contiguous nucleotide sequence are phosphorothioate intemucleoside linkages.

13. The oligonucleotide according to claim 1, wherein the oligonucleotide comprises a sequence selected from the group consisting of: SEQ ID NO: 3, SEQ ID NO: 4, SEQ ID NO: 5, SEQ ID NO: 6, and SEQ ID NO: 7.

14. The oligonucleotide according to claim 1, wherein the oligonucleotide is admixed with a pharmaceutically acceptable diluent or carrier.

15. A method for modulating the expression of SMN2 in a cell expressing SMN2, the method comprising the step of administering an oligonucleotide of claim 1 to the cell or a population of cells.

16. The method of claim 15, wherein the population of cells is in a patient having a pathological condition associated with a reduction in motor neuron or neuromuscular junction numbers or with a disruption of motor neurons or neuromuscular junctions, and wherein the modulation of the expression of SMN2 is a treatment for, or a prevention of, the pathological condition of the recipient patient.

17. A method of increasing a steady-state level of an mRNA encoding SMN2 in a subject in need thereof, the method comprising providing to the subject a therapeutically effective amount of an oligonucleotide of claim 1, wherein the oligonucleotide promotes the transcription of mRNA encoding SMN2, or inhibits degradation of an mRNA encoding SMN2 in cells of the subject in need thereof.

18. The method of claim 17, wherein the subject has a condition associated with a reduction in motor neuron or neuromuscular junction numbers, or a condition associated with disruption of motor neurons or neuromuscular junctions.

19. The method of claim 17, wherein the subject has spinal muscular atrophy, has 5q spinal muscular atrophy, is a geriatric subject, has an acute muscle injury, or has a chronic muscle injury.

20. The method of claim 17, wherein the oligonucleotide is co-administered with a splice-switching promoter, and wherein the oligonucleotide and the splice-switching promoter are administered to the patient simultaneously or sequentially.

21. The method of claim 20, wherein the splice-switching promoter promotes exon 7 inclusion in SMN2 mRNA.

22. The method of claim 20, wherein the splice-switching promoter is selected from the group consisting of: nusinersen, risdiplam, branaplam, or an oligonucleotide.

23. The method of claim 17, wherein the oligonucleotide comprises the nucleotide sequence of SEQ ID No: 14.

24. The method of claim 23, wherein the splice-switching promoter is nusinersen.

25. The method of claim 17, wherein the oligonucleotide is provided directly to the central nervous system of the subject.

25. The method of claim 17, wherein the oligonucleotide is provided directly to the cerebrospinal fluid of the subject.

26. The method of claim 17, wherein the oligonucleotide is provided to the subject by injection or by intrathecal injection.

27. The method of claim 23, wherein the splice-switching promoter is provided by injection.

28. The method of claim 23, wherein the splice-switching promoter is selected from risdiplam and branaplam, and is provided by oral administration.

29. The method of claim 17, wherein the oligonucleotide is provided in an amount of between about 5 mg to about 50 mg or between about 10 mg and about 15 mg.


SEQUENCE LISTING (18 sequences)

SEQ ID NO: 1 - DNA, 33 bp, Artificial sequence
  SMN2 5′UTR target region
ccacaaatgtgggagggcgataaccactcgtag

SEQ ID NO: 2 - DNA, 33 bp, Artificial sequence
  SMN2 5′UTR target region complement
ctacgagtggttatcgccctcccacatttgtgg

SEQ ID NO: 3 - DNA, 24 bp, Artificial sequence
  Antisense Oligonucleotide AON#1
gttatcgccctcccacatttgtgg

SEQ ID NO: 4 - DNA, 24 bp, Artificial sequence
  Antisense Oligonucleotide AON#2
tggttatcgccctcccacatttgt

SEQ ID NO: 5 - DNA, 24 bp, Artificial sequence
  Antisense Oligonucleotide AON#3
agtggttatcgccctcccacattt

SEQ ID NO: 6 - DNA, 24 bp, Artificial sequence
  Antisense Oligonucleotide AON#4
cgagtggttatcgccctcccacat

SEQ ID NO: 7 - DNA, 24 bp, Artificial sequence
  Antisense Oligonucleotide AON#5
tacgagtggttatcgccctcccac

SEQ ID NO: 8 - DNA, 24 bp, Artificial sequence
  Antisense Oligonucleotide AON#1 complement
ccacaaatgtgggagggcgataac

SEQ ID NO: 9 - DNA, 24 bp, Artificial sequence
  Antisense Oligonucleotide AON#2 complement
acaaatgtgggagggcgataacca

SEQ ID NO: 10 - DNA, 32 bp, Artificial sequence
  Antisense Oligonucleotide AON#3 complement
  misc_feature (7)..(7): n is a, c, g, or t
artcasncaaatgtgggagggcgataaccact

SEQ ID NO: 11 - DNA, 24 bp, Artificial sequence
  Antisense Oligonucleotide AON#4 complement
atgtgggagggcgataaccactcg

SEQ ID NO: 12 - DNA, 24 bp, Artificial sequence
  Antisense Oligonucleotide AON#5 complement
gtgggagggcgataaccactcgta

SEQ ID NO: 13 - DNA, 25 bp, Artificial sequence
  Non-targeting Control oligonucleotide
cctcttacctcagttacaatttata

SEQ ID NO: 14 - DNA, 18 bp, Artificial sequence
  Splice-switching oligonucleotide
tcactttcataatgctgg

SEQ ID NO: 15 - DNA, 20 bp, Artificial sequence
  Total SMN qForward Primer
gcgatgattctgacatttgg

SEQ ID NO: 16 - DNA, 20 bp, Artificial sequence
  Total SMN qReverse Primer
ggaagctgcagtattcttct

SEQ ID NO: 17 - DNA, 24 bp, Artificial sequence
  GAPDH qForward Primer
ctcaacgaccactttgtcaagctc

SEQ ID NO: 18 - DNA, 22 bp, Artificial sequence
  GAPDH qReverse Primer
tcttactccttggaggccatgt